(19) United States
(12) Patent Application Publication
(10) Pub. No.: US 2015/0167089 A1
(43) Pub. Date: Jun. 18, 2015

(54) Gene Mutations for the Diagnosis of Arthrogryposis Multiplex Congenita and Congenital Peripheral Neuropathies Disease

(71) Applicants: INSERM (Institut National de la Sante et de la Recherche Medicale), Paris (FR); Association Francaises Contre Les Myopathies, Paris (FR)

(72) Inventor: Judith Melki, Le Kremlin-Bicetre Cedex (FR)

(21) Appl. No.: 14/575,518

(22) Filed: Dec. 18, 2014

Related U.S. Application Data
(63) Continuation-in-part of application No. PCT/IB2013/002937, filed on Dec. 18, 2013.

(51) Int. Cl. C12Q 1/68

(52) U.S. Cl. CPC C12Q 1/6883; C12Q 2600/156; C12Q 2600/106

(57) ABSTRACT

The present invention relates to a method of identifying a subject having or at risk of having or developing arthrogryposis multiplex congenita and/or congenital peripheral neuropathy, comprising determining, in a sample obtained from said subject, the presence or absence of a single nucleotide variant (SNV) in CNTNAP1, ADCY6, LGI4 or LMOD3 genes

### FIELD OF THE INVENTION

The invention is in the field of arthrogryposis multiplex congenita (AMC) and/or congenital peripheral neuropathy diagnosis and therapy. In particular, the invention relates to specific mutations (or Single Nucleotide Variant, SNV) in human genes responsible for arthrogryposis multiplex congenita and congenital peripheral neuropathies.

### BACKGROUND OF THE INVENTION

Arthrogryposis multiplex congenita (AMC) is characterized by congenital contractures of at least two distinct joints of the body. The overall incidence is 1 in 3000 of live births.^{1,2} Some non genetic factors may cause AMC such as mechanical limitation of fetal movements or maternal autoimmune myasthenia. A number of genetic syndromes including AMC phenotype, collectively referred to as syndromic AMC, have been described in several conditions.^{3,4} Non-syndromic or isolated AMC are the direct consequence of fetal akinesia/hypokinesia sequence which may lead, in addition to AMC, to pterygia, lung hypoplasia, diaphragmatic defect or cleft palate. Isolated AMC are genetically heterogeneous. Mutations of genes encoding components of the neuromuscular junctions including CHRNG, CHRNE, CHRNA1, CHRND, CHRNB1, RAPSN, CHAT or DOK7 are responsible for multiple pterygium syndromes. Fetal motor neuron diseases may also result in lethal congenital contractures caused by mutations of GLE1, PIP5K1C or ERBB3 genes. AMC also occasionally occurs in type I spinal muscular atrophy (SMA) caused by mutations of SMN1 or congenital SMA linked to TRPV4 mutations. More recently, mutations of ECEL1 have been reported in distal AMC.^{5,6} Congenital myopathies associated with distal AMC may be caused by mutations of TPM2, MYH2, MYH3, MYH8, MYH13, TNNI2, TNNT3 or MYBPC1 genes. Congenital myotonic dystrophy caused by abnormal triplet expansion of the DMPK gene, nemalin myopathy linked to ACTA1, TPM2 or NEB, minicore myopathy linked to RYR1 mutations or more recently SYNE1 mutation have all been reported in non syndromic AMC. Collectively, non-syndromic AMCs include a large spectrum of diseases of motor neurons, neuromuscular junctions, or skeletal muscle.

The difficulty in establishing a genetic diagnosis for AMC patients is likely due to the high genetic heterogeneity and/or to some not yet identified disease genes. Moreover, there is a lack of suitable screening methods of all known AMC genes

Identifying additional risk genes and understanding their mechanisms of action through experimental and functional studies represent current challenges and are critical for the use of this knowledge in disease prevention or therapy.

Therefore, there is a need for identifying genes that provide a more accurate diagnosis/prognosis of AMC.

In order to gain insight into the underlying cause of these diseases, inventors took advantage of the added value of whole genome scanning using SNP microarrays alone or combined with whole exome sequencing in a cohort of 33 families with undiagnosed non-syndromic AMC

Here, mutations in known disease genes were identified by the inventors in 19 families (57%) and four new genes (CNTNAP1, LGI4, ADCY6 and LMOD3) were found to be mutated in 7 families (21%), three of them being involved in axoglial interaction.

Consequently, the inventors also describe identification of new AMC-causing genes, present in ˜20% of AMC families. Screening for these genes should improve the clinical outcome for young members of these families by allowing early detection and appropriate clinical management of AMC from its earliest stages (even at embryo or fetal stage).

### SUMMARY OF THE INVENTION

A first object of the invention is a method of identifying a subject having or at risk of having or developing an arthrogryposis multiplex congenita (AMC) and/or congenital peripheral neuropathy, comprising determining, in a sample obtained from said subject, the presence or absence of a single nucleotide variant (SNV) located in CNTNAP1, LGI4, ADCY6 and LMOD3 genes.

In a preferred embodiment, the SNV is selected from the group consisting of CNTNAP1: NM_{—}003632:c.2901_{—}2902del, CNTNAP1: NM_{—}003632:c.3009_{—}3010 insT; CNTNAP1: NMF_{—}003632:c.2993-2_{—}2994del, LGI4: NM_{—}139284:c.G793A, ADCY6: NM_{—}015270:c.C3346T, LMOD3: NM_{—}198271:c.135_{—}136insC and wherein:
the presence of the allele (del) of CNTNAP1: NM_{—}003632:c.2901_{—}2902del indicates an increased risk of having or developing an arthrogryposis multiplex congenita and/or congenital peripheral neuropathy;
the presence of the allele (insT) of CNTNAP1: NM_{—}003632:c.3009_{—}3010 insT indicates a increased risk of having or developing an arthrogryposis multiplex congenita and/or congenital peripheral neuropathy;
the presence of the allele (del) of CNTNAP1: NM_{—}003632:c.2993-2_{—}2994del indicates a increased risk of having or developing an arthrogryposis multiplex congenita and/or congenital peripheral neuropathy;
the presence of the allele (A) of LGI4: NM_{—}139284c.G793A indicates a increased risk of having or being at risk of having or developing an arthrogryposis multiplex congenita and/or congenital peripheral neuropathy;
the presence of the allele (T) of ADCY6: NM_{—}015270:c.C3346T indicates a increased risk of having or developing an arthrogryposis multiplex congenita and/or congenital peripheral neuropathy;
the presence of the allele (insC) of LMOD3: NM_{—}198271:c.135_{—}136 insC indicates a increased risk of having or developing an arthrogryposis multiplex congenita and/or congenital peripheral neuropathy.

A second object of the invention is a kit for identifying whether a subject has or is at risk of having or developing an arthrogryposis multiplex congenita and/or congenital peripheral neuropathy, comprising:
at least a means for detecting the SNV selected from the group consisting of CNTNAP1: NM_{—}003632:c.2901_{—}2902del, CNTNAP1: NM_{—}003632:c.3009_{—}3010insT; CNTNAP1: NM_{—}003632:c.2993-2_{—}2994del, LGI4: NM_{—}139284c.G793A, ADCY6: NM_{—}015270:c.C3346T, LMOD3: NM_{—}198271:c.135_{—}136insC and
instructions for use.

A third object of the invention is a nuclease for use in treating arthrogryposis multiplex congenita and/or congenital peripheral neuropathy and/or preventing progression of arthrogryposis multiplex congenita and/or congenital peripheral neuropathy in a patient, wherein the presence of SNV in CNTNAP1, LGI4, ADCY6 and LMOD3 genes in a sample previously obtained from said patient, have been detected by a method of the invention previously described.

### DETAILED DESCRIPTION OF THE INVENTION

### Definitions

Throughout the specification, several terms are employed and are defined in the following paragraphs.

The term “arthrogryposis multiplex congenita” is a medical condition which is characterized by congenital contractures of at least two distinct joints of the body. Some non genetic factors may cause AMC such as mechanical limitation of fetal movements or maternal autoimmune myasthenia. A number of genetic syndromes including AMC phenotype, collectively referred to as syndromic AMC, have been described in several conditions.^{3,4} Non-syndromic or isolated AMC are the direct consequence of fetal akinesia/hypokinesia sequence which may lead, in addition to AMC, to pterygia, lung hypoplasia, diaphragmatic defect or cleft palate. Isolated AMC are genetically heterogeneous

According to the method of the invention Arthrogryposis multiplex congenita or “AMC” means “syndromic AMC” and “non syndromic AMC”.

Patients carrying deleterious mutations in CNTNAP1, ADCY6 and LGI4 have a AMC caused by a peripheral hypo or amyelinic neuropathy as determined by electron microscopic examination of the peripheral nerve. This neuropathy is responsible for reduced motor nerve conduction velocity causing reduced fetal mobility and AMC. Therefore, mutations of these genes are responsible for novel cause of congenital peripheral neuropathies.

Accordingly, the method of the present invention encompasses diagnostic method for congenital peripheral neuropathy.

A “peripheral neuropathy” is defined as a disorder of the peripheral nervous system, involving at least one of its motor, sensory or autonomic components. Each component includes the neuron cell body, its axons and the myelinating or non myelinating Schwann cells wrapping the axon. These components can be injured in the spinal cord anterior horns (motor neurons), in the dorasal root ganglia (sensory neurons) in the autonomic ganglia (autonomic neurons) and in the peripheral nerve including roots, plexus and nerves proper in which the axons circulate (see Dyck, Thomas, Lambert, Bunge. Peripheral Neuropathy. Vol. II. W.B. Saunders Co, Philadelphia, Pa. 1984).

CNTNAP1, LGI4, ADCY6 and LMOD3 Genes

The term “CNTNAP1” also known as “contactin associated protein 1” and “P190”; “CASPR”; “NRXN4”; CNTNAP″ means contactin associated protein 1 (NM_{—}003632 NP_{—}003623) which is predominantly expressed in brain and nerve and seems to play a role in the formation of functional distinct domains critical for saltatory conduction of nerve impulses in myelinated nerve fibers. CNTNAP1 seems to demarcate the paranodal region of the axo-glial junction. In association with contactin may have a role in the signaling between axons and myelinating glial cells. The whole sequence of human CNTNAP1 gene is referenced as Gene ID: 8506.

The term “LGI4” also known as “leucine-rich repeat LGI family, member 4” and “LGIL3” is a protein-coding gene and means LGI4 protein (NM_{—}139284 NP_{—}644813) which seems to play a role in the glial cell proliferation, myelination in peripheral nervous and neuron maturation. The whole sequence of human LGI4 gene is referenced as Gene ID: 163175.

The term “ADCY6” also known as “adenylate cyclase 6” and “AC6” means adenylate cyclase 6 protein (NM_{—}015270 NP_{—}056085) which is a membrane-associated enzyme and catalyzes the formation of the secondary messenger cyclic adenosine monophosphate (cAMP). The expression of this gene is found in normal thyroid and brain tissues. Alternative splicing generates 2 transcript variants. The whole sequence of human ADCY6 gene is referenced as Gene ID: 112.

The term “LMOD3” also known as “Leiomodin 3 (Fetal)” and “Leiomodin Fetal form” means Leiomodin 3 protein (NM_{—}198271 NP_{—}938012) which have unknown function and unknown diseases associated with LMOD3. LMOD3 belongs to the tropomodulin family (with conserved domains). The whole sequence of human LMOD3 gene is referenced as Gene ID: 56203.

“Risk” in the context of the present invention, relates to the probability that an event will occur over a specific time period, as in the conversion to AMC and/or congenital peripheral neuropathy disease, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no conversion. Alternative continuous measures which may be assessed in the context of the present invention include time to AMC and/or congenital peripheral neuropathy disease conversion and therapeutic AMC and/or congenital peripheral neuropathy disease conversion risk reduction ratios.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to a AMC condition and/or congenital peripheral neuropathy condition or to one at risk of developing a AMC and/or congenital peripheral neuropathy disease. Risk evaluation can also comprise prediction of future clinical parameters, traditional laboratory risk factor values, or other indices of AMC and/or congenital peripheral neuropathy diseases, such as dopamine level detection, cellular population determination in peripheral tissues, in serum or other fluid (i.e. cerebrospinal fluid), either in absolute or relative terms in reference to a previously measured population. The methods of the present invention may be used to make continuous or categorical measurements of the risk of conversion to AMC and/or congenital peripheral neuropathy diseases, thus diagnosing and defining the risk spectrum of a category of subjects defined as being at risk for a AMC and/or congenital peripheral neuropathy diseases. In the categorical scenario, the invention can be used to discriminate between normal and other subject cohorts at higher risk for AMC and/or congenital peripheral neuropathy diseases. In other embodiments, the present invention may be used so as to help to discriminate those having AMC and/or congenital peripheral neuropathy diseases from normal.

“Clinical parameters or indicia” encompasses all non-sample or non-analyte biomarkers of subject health status or other characteristics, such as, without limitation, age (Age), geographical origin (Origin), gender (Sex), family history (FamHX), height (HT), weight (WT), waist (Waist) and body-mass index (BMI), as well as others such as clinical cardinal signs of AMC disease (like congenital contractures of at least two distinct joints of the body).

A “sample” in the context of the present invention is a biological sample isolated from a subject and can include, by way of example and not limitation, bodily fluids and/or tissue extracts such as homogenates or solubilized tissue obtained from a subject. Tissue extracts are obtained routinely from tissue biopsy and autopsy material. Bodily fluids useful in the present invention include blood, urine, saliva or in case of fetus, amniotic fluid or chorionic villy or any other bodily secretion or derivative thereof. In a preferred embodiment, the sample to be tested is saliva or blood. As used herein “blood” includes whole blood, plasma, serum, circulating epithelial cells, constituents, or any derivative of blood.

In a preferred embodiment the sample is a blood sample or amniotic fluid or chorionic villy.

According to the invention, CNTNAP1, LGI4, ADCY6 and LMOD3 mutations are genomic variants and are detected by using any type of body cell. In a preferred embodiment the cell is a blood cell fetal cells from amniotic fluid or chorionic villy cell.

According to the invention, the sample comprises CNTNAP1, LGI4, ADCY6 and LMOD3 nucleic acid, wherein CNTNAP1, LGI4, ADCY6 and LMOD3 nucleic acid is genomic DNA.

A “subject” in the context of the present invention is preferably a human.

The term “Allele” has the meaning which is commonly known in the art, that is, an alternative form of a gene (one member of a pair) that is located at a specific position on a specific chromosome which, when translated results in functional or dysfunctional (including non-existent) gene products.

The term “mutation” or “allelic variant” means a sequence variation of a gene. Allelic variants can be found in the exons, introns, untranslated regions of the gene, or in the sequences that control expression of the gene. Complete gene sequencing often identifies numerous allelic variants (sometimes hundreds) for a given gene. The significance of allelic variants is often unclear until further study of the genotype and corresponding phenotype occurs in a sufficiently large population.

The term “Single nucleotide variant” or “SNV” refers to a type of DNA variation of a single base pair or insertions/deletions. There are millions of SNVs in the human genome. Most commonly, these variations are found in coding sequences of genes, non-coding regions of genes, or in intergenic regions between genes. When SNVs occur within a gene or in a regulatory region near a gene, they may play a more direct role in disease by affecting the gene's function.

The SNV pertaining to the invention are unknown (known sequences are publicly available from the data base http://www.ncbi.nlm.nih.gov/SNP/). The mutations studied are described here after:

| | Chromosome | SNV Chr | | | Aminoacid |
|---|---|---|---|---|---|
| HGVS name | position | Position | Gene | Type | Change |
| NM_003632:exon18: | 17q21 | 17: 40845463 | CNTNAP1 | frameshift | P967PfsX12 |
| c.2901_2902del | | | | | (exon 18) |
| NM_003632:exon19: | 17q21 | 17: 40847555 | CNTNAP1 | frameshift | F1003fs (exon |
| c.3009_3010insT | | | | | 19) |
| NM_003632:intron18- | 17q21 | 17: 40847537 | CNTNAP1 | frameshift | I999WfsX5 |
| exon19: | | | | | (intron18- |
| c.2993-2_2994del | | | | | exon19) |
| NM_139284:exon7:c. | 19q13.11 | 19: 35617757 | LGI4 | missense | A265T (exon 7) |
| G793A | | | | (d; 0.96) | |
| NM_015270:exon20: | 12q12- | 12: 49162755 | ADCY6 | missense | R1116C (exon |
| c.C3346T | q13 | | | (d; 1) | 20) |
| NM_198271:exon1:c. | 3p14.1 | 3: 69171403 | LMOD3 | frameshift | D45fs (exon 1) |
| 135_136insC | | | | | |

### Diagnostic Method

The present inventors have assayed for a statistical link between specific variants located at CNTNAP1, LGI4, ADCY6 and LMOD3 genes and arthrogryposis multiplex congenita (AMC) using a cohort of AMC disease families and, patients. More precisely, the present inventors have assayed for a statistical link between specific SNV contained in chromosome 17q21 locus, 19q13.12; 19q13.11 locus, 12q12-q13 locus and 3p14 locus of AMC patients (multiplex families and cases) and controls.

By genetic mapping, SNP genotyping and whole exome sequencing in AMC patients belonging to 33 multiplex and/or consanguineous undiagnosed AMC families, the inventors identified known AMC genes, but have found pathogenic mutations in four new genes (CNTNAP1, LGI4, ADCY6 and LMOD3). More precisely, the present inventors have identified specific SNVs contained in the sequence of the CNTNAP1, LGI4, ADCY6 and LMOD3 genes, and associated with arthrogryposis multiplex congenita in the corresponding patients.

As disclosed in the examples herein, the inventors have screened DNA blood or tissue samples of a well characterized cohort of AMC families and patients to assess the genomic effects of single nucleotide variants (SNVs) at different loci. Evidence that AMC is caused by 17q21, 19q13.12; 19 q13.11, 12q12-q13 and 3p14 locus mutations in CNTNAP1, LGI4, ADCY6 and LMOD3 genes in several families and patients have been provided.

More precisely, the inventors have now identified specific SNV biallelics marker located in CNTNAP1, LGI4, ADCY6 and LMOD3 genes, wherein the SNV biallelic marker selected from the group consisting of CNTNAP1:c.2901_{—}2902del, CNTNAP1: c.3009_{—}3010insT; CNTNAP1:c.2993-2_{—}2994del, LGI4:c.G793A, ADCY6:c.C3346T, LMOD3:c.135_{—}136insC, was not only associated with AMC but cause AMC.

Indeed, the inventors have also performed transmission electron microscopy (TEM) of sciatic nerve in the index cases which revealed severe abnormalities of both nodes of Ranvier width and myelinated axons in AMC patients with mutations of CNTNAP1 and ADCY6. In patient with mutation of LMOD3, the morphology of the muscle biopsy revealed the presence of multiple rods compatible with the diagnosis of nemalin myopathy. TEM confirmed the presence of rods with their characteristic lattice pattern associated with small dense rectangular structures suggesting Z bands disruption.

In addition, the inventors have also performed functional analysis of ADCY6 knockdowns in zebrafish model, and demonstrated severe and specific defects in peripheral myelin in spite of the presence of Schwann cells similar to those observed in patients and during early development (at embryos stage).

These results support that the ADCY6 mutations at exon 20, in particular the R1116C ADCY6 mutation (SNV c.C3346T) and other CNTNAP1, LGI4 and LMOD3 mutations which are found in up to 21% of familial AMC cases in this cohort, are strong predictor of AMC disease occurrence during human embryo development. Furthermore, ADCY6 mutations at exon 20 (as CNTNAP1 mutations at exon 18 and 19, LGI4 mutations at exon 7, LMOD3 mutations at exon 1) could be a therapeutic target in (young) subjects or fetuses.

A first object of the invention is a method of identifying a subject having or at risk of having or developing a AMC and/or congenital peripheral neuropathy disease, comprising determining, in a sample obtained from said subject, the presence or absence of single nucleotide variant (SNV) located in CNTNAP1, LGI4, ADCY6 or LMOD3 genes.

In particular embodiment the SNV is selected from the group consisting of CNTNAP1: NM_{—}003632:c.2901_{—}2902del, CNTNAP1: NM_{—}003632:c.3009_{—}3010insT; CNTNAP1: NM_{—}003632:c.2993-2_{—}2994del, LGI4: NM_{—}139284:c.G793A, ADCY6: NM_{—}015270:c.C3346T, LMOD3: NM_{—}198271:c.135_{—}136insC and wherein:
the presence of the allele (del) of CNTNAP1: NM_{—}003632:c.2901_{—}2902del indicates an high risk of having or developing an arthrogryposis multiplex congenita and/or congenital peripheral neuropathy;
the presence of the allele (insT) of CNTNAP1: NM_{—}003632:c.3009_{—}3010insT indicates a high risk of having or developing an arthrogryposis multiplex congenita and/or congenital peripheral neuropathy;
the presence of the allele (del) of CNTNAP1: NM_{—}003632:c.2993-2_{—}2994del indicates a high risk of having or developing an arthrogryposis multiplex congenita and/or congenital peripheral neuropathy;
the presence of the allele (A) of LGI4: NM_{—}139284:c.G793A indicates a high risk of having or being at risk of having or developing an arthrogryposis multiplex congenita and/or congenital peripheral neuropathy;
the presence of the allele (T) of ADCY6: NM_{—}015270:c.C3346T indicates a high risk of having or developing an arthrogryposis multiplex congenita and/or congenital peripheral neuropathy;
the presence of the allele (insC) of LMOD3: NM_{—}198271:c.135_{—}136insC indicates a high risk of having or developing an arthrogryposis multiplex congenita and/or congenital peripheral neuropathy.

According to the invention, the presence of the allele (del) of CNTNAP1: NM_{—}003632:c.2901_{—}2902del indicates a high risk of having or developing AMC and/or congenital peripheral neuropathy disease. As shown in the examples, allele (del) of CNTNAP1: NM_{—}003632:c.2901_{—}2902del was detected in family A641 so significantly increases the risk of AMC compared with controls.

According to the invention, the presence of the allele ((insT) of CNTNAP1: NM_{—}003632:c.3009_{—}3010insT indicates an high risk of having or developing AMC and/or congenital peripheral neuropathy disease. As shown in the examples, allele (insT) of CNTNAP1: NM_{—}003632:c.3009_{—}3010insT was detected in families K182 and K199 so significantly increases the risk of AMC compared with controls

According to the invention, the presence of the allele (del) of CNTNAP1: NM_{—}003632:c.2993-2_{—}2994del indicates an high risk of having or developing AMC and/or congenital peripheral neuropathy disease. As shown in the examples, allele (del) of CNTNAP1: NM_{—}003632:c.2993-2_{—}2994del was detected in family B207 so significantly increases the risk of AMC compared with controls.

According to the invention, the presence of the allele (A) of LGI4: NM_{—}139284c.G793A indicates an high risk of having or developing AMC and/or congenital peripheral neuropathy disease. As shown in the examples, allele (A) of SNV LGI4: NM_{—}139284c.G793A was detected in family A633 so significantly increases the risk of AMC compared with controls

According to the invention, the presence of the allele (T) of ADCY6: NM_{—}015270:c.C3346T indicates an high risk of having or developing AMC and/or congenital peripheral neuropathy disease. As shown in the examples, allele (T) of ADCY6: NM_{—}015270:c.C3346T was detected in family A649 so significantly increases the risk of AMC compared with controls

According to the invention, the presence of the allele (insC) of LMOD3: NM_{—}198271:c.135_{—}136insC indicates an high risk of having or developing AMC and/or congenital peripheral neuropathy disease. As shown in the examples, allele (insC) of SNP LMOD3: NM_{—}198271:c.135_{—}136insC was detected in family K177 so significantly increases the risk of AMC compared with controls

In one embodiment of the invention, the method of identifying a subject having or at risk of having or developing a AMC disease, comprising determining the presence or absence of an rare allelic variant located in CNTNAP1, LGI4, ADCY6 and LMOD3 genes in a blood, amniotic fluid, chorionic villi or tissue sample obtained from said subject.

In another embodiment, said subject may also be one that is asymptomatic for the arthrogryposis multiplex congenita. As used herein, an “asymptomatic” subject refers to a subject that does not exhibit AMC symptoms, which are diagnosed, according to internationally validated criteria (Hall, J. G. Genetic aspects of arthrogryposis. (1985) Clin. Orthop., 194, 44-53).

In another embodiment of the invention, said subject may be one that is at risk of having or developing an AMC and/or congenital peripheral neuropathy disease, as defined by clinical indicia such as for example: age, gender, clinical marker (like congenital contractures of at least two distinct joints of the body), family history of AMC and/or congenital peripheral neuropathy disease.

As shown by the inventors, targeting previously known AMC genes, and the identification of four novel genes in this study should now lead to a molecular diagnosis in more than 75% of AMC cases.

Accordingly, in one embodiment of the invention, the method of identifying a subject having or at risk of having or developing a AMC disease as previously described, comprises a further step by determining the presence or absence of a single nucleotide variant (SNV) located in genes previously known to be responsible for AMC or neuromuscular disorders (CHRNG, CHRNA1, CHRND, CHRNB1, DOK7, RAPSN, CHAT, GLE1, PIP5K1C, ERBB3, SMN1, TRPV4, ECEL1, TPM2, MYH2, MYH3, MYH8, TNNI2, TNNT3, MYBPC1, DMPK, ACTA1, TPM2, NEB, RYR1, SYNE1, RBBP8 or new genes (CNTNAP1, ADCY6, LMOD3 and LGI4) described here in table 1 in a sample obtained from said subject.

According to the invention, the determination of the presence or absence of said SNVs may be determined by DNA sequencing, PCR analysis or any genotyping method known in the art. Examples of such methods include, but are not limited to, chemical assays such as allele specific hybridation, primer extension, allele specific oligonucleotide ligation, sequencing, enzymatic cleavage, flap endonuclease discrimination; and detection methods such as fluorescence, chemiluminescence, and mass spectrometry.

For example, the presence or absence of said variant may be detected in a DNA sample, preferably after amplification. For instance, the isolated DNA may be subjected to amplification by polymerase chain reaction (PCR), using specific oligonucleotide primers that are specific for the SNV or that enable amplification of a region flanking the SNV. According to a first alternative, conditions for primer annealing may be chosen to ensure specific amplification; so that the appearance of an amplification product be a diagnostic of the presence of the SNV according to the invention. Otherwise, DNA may be amplified, after which a mutated site may be detected in the amplified sequence by hybridization with a suitable probe or by direct sequencing, or any other appropriate method known in the art.

Actually numerous strategies for genotype analysis are available (Cooper et al., 1991; Grompe, 1993). Briefly, the nucleic acid molecule may be tested for the presence or absence of a restriction site. When a base polymorphism creates or abolishes the recognition site of a restriction enzyme, this allows a simple direct PCR genotype of the polymorphism. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al., 1997). Direct sequencing may be accomplished by any method, including without limitation chemical sequencing, using the Maxam-Gilbert method; by enzymatic sequencing, using the Sanger method; mass spectrometry sequencing; sequencing using a chip-based technology; and real-time quantitative PCR. Preferably, DNA from a subject is first subjected to amplification by polymerase chain reaction (PCR) using specific amplification primers. However several other methods are available, allowing DNA to be studied independently of PCR, such as the rolling circle amplification (RCA), the InvaderTMassay, or oligonucleotide ligation assay (OLA). OLA may be used for revealing base polymorphisms. According to this method, two oligonucleotides are constructed that hybridize to adjacent sequences in the target nucleic acid, with the join sited at the position of the polymorphism. DNA ligase will covalently join the two oligonucleotides only if they are perfectly hybridized to one of the allele.

Therefore, short DNA sequences, in particular oligonucleotide probes or primers, according to the present invention include those which specifically hybridize the one of the allele of the polymorphism.

Oligonucleotide probes or primers may contain at least 10, 15, 20 or 30 nucleotides. Their length may be shorter than 400, 300, 200 or 100 nucleotides.

According to the invention, the determination of the presence or absence of said SNVs may also be determined by detection or not of the mutated CNTNAP1, LGI4, ADCY6 and LMOD3 protein(s) (i.e. CNTNAP1: P967PfsX12, F1003fs or I999WfsX5) by any method known in the art. The presence of the protein of interest may be detected using standard electrophoretic and immunodiagnostic techniques, including immunoassays such as competition, direct reaction, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith. Labels are known in the art that generally provide (either directly or indirectly) a signal. As used herein, the term “labelled” with regard to the antibody or aptamer, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or indocyanine (Cy5), to the antibody or aptamer, as well as indirect labelling of the probe or antibody (e.g., horseradish peroxidise, HRP) by reactivity with a detectable substance. An antibody or aptamer may be also labelled with a radioactive molecule by any method known in the art. For example, radioactive molecules include but are not limited radioactive atom for scintigraphic studies such as I123, I124, In111, Re186 and Re188. The aforementioned assays generally involve separation of unbound protein in a liquid phase from a solid phase support to which antigen-antibody complexes are bound. Solid supports which may be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, etc.

More particularly, an ELISA method may be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

Alternatively, an immunohistochemistry (IHC) method may be used. IHC specifically provides a method of detecting a target in a biological sample or tissue specimen in situ. The overall cellular integrity of the sample is maintained in IHC, thus allowing detection of both the presence and location of the target of interest. Typically a biological sample is fixed with formalin, embedded in paraffin and cut into sections for staining and subsequent inspection by light microscopy. Current methods of IHC use either direct labelling or secondary antibody-based or hapten-based labelling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan).

In particular embodiment, a tissue section (e.g. a tissue sample or biopsy) may be mounted on a slide or other support after incubation with antibodies directed against (the) protein(s) encoded by CNTNAP1, LGI4, ADCY6 and LMOD3 gene(s) with SNVs located at the different exon. Then, microscopic inspections in the sample mounted on a suitable solid support may be performed. For the production of photomicrographs, sections comprising samples may be mounted on a glass slide or other planar support, to highlight by selective staining the presence of the protein of interest. Therefore IHC samples may include, for instance: (a) preparations comprising cell samples (b) fixed and embedded said cells and (c) detecting the protein of interest in said cell samples. In some embodiments, an IHC staining procedure may comprise steps such as: cutting and trimming tissue, fixation, dehydration, paraffin infiltration, cutting in thin sections, mounting onto glass slides, baking, deparaffination, rehydration, antigen retrieval, blocking steps, applying primary antibodies, washing, applying secondary antibodies (optionally coupled to a suitable detectable label), washing, counter staining, and microscopic examination.

Accordingly additional object of the invention relates to an in vitro method for diagnosing and/or prognosis of AMC and/or congenital peripheral neuropathy disease in a subject, said method comprising determining, in a sample obtained from said subject, the presence or absence of single nucleotide variants (SNVs) located in CNTNAP1, LGI4, ADCY6 and LMOD3 genes.

In a preferred embodiment, the SNV is selected from the group consisting of CNTNAP1: NM_{—}003632:c.2901_{—}2902del, CNTNAP1: NM_{—}003632:c.3009_{—}3010insT; CNTNAP1: NM_{—}003632:c.2993-2_{—}2994del, LGI4: NM_{—}139284:c.G793A, ADCY6: NM_{—}015270:c.C3346T, LMOD3: NM_{—}198271:c.135_{—}136insC and wherein:
the presence of the allele (del) of CNTNAP1: NM_{—}003632:c.2901_{—}2902del is indicative of an arthrogryposis multiplex congenita and/or congenital peripheral neuropathy or that the diseases will evolved in worse manner;
the presence of the allele (insT) of CNTNAP1: NM_{—}003632:c.3009_{—}3010insT is indicative of an arthrogryposis multiplex congenita and/or congenital peripheral neuropathy or that the diseases will evolved in worse manner;
the presence of the allele (del) of CNTNAP1: NM_{—}003632:c.2993-2_{—}2994del is indicative of an arthrogryposis multiplex congenita and/or congenital peripheral neuropathy or that the diseases will evolved in worse manner;
the presence of the allele (A) of LGI4: NM_{—}139284:c.G793A is indicative of an arthrogryposis multiplex congenita and/or congenital peripheral neuropathy or that the diseases will evolved in worse manner;
the presence of the allele (T) of ADCY6: NM_{—}015270:c.C3346T is indicative of an arthrogryposis multiplex congenita and/or congenital peripheral neuropathy or that the diseases will evolved in worse manner;
the presence of the allele (insC) of LMOD3: NM_{—}198271:c.135_{—}136 insC indicates a high risk of having or developing an arthrogryposis multiplex congenita.

The “diagnosis” means the identification of the condition or the assessment of the severity of the disease or that the diseases will evolved in worse manner.

The term “prognosis” means the assessment of the outcome of the disease, i.e. to determine the evolution of the condition, and the risk of worsening.

### Kit of the Invention

A second object of the invention is a kit for identifying whether a subject has or is at risk of having or developing an AMC and/or congenital peripheral neuropathy disease, comprising:
at least a means for detecting the SNV located in CNTNAP1, LGI4, ADCY6 and LMOD3 genes. and
instructions for use.

In one embodiment of the invention the kit for identifying whether a subject has or is at risk of having or developing an AMC and/or congenital peripheral neuropathy disease, comprising:
at least a means for detecting the SNV selected from the group consisting of CNTNAP1: NM_{—}003632:c.2901_{—}2902del, CNTNAP1: NM_{—}003632:c.3009_{—}3010insT; CNTNAP1: NM_{—}003632:c.2993-2_{—}2994del, LGI4: NM_{—}139284:c.G793A, ADCY6: c.C3346T, LMOD3: NM_{—}198271:c.135_{—}136insC and
instructions for use.

In one embodiment of the invention, the kit for identifying whether a subject has or is at risk of having or developing an AMC and/or congenital peripheral neuropathy disease, comprising:
at least one primer and/or at least one probe for amplification of a sequence comprising a SNV consisting of CNTNAP1: NM_{—}003632:c.2901_{—}2902del, CNTNAP1: NM_{—}003632:c.3009_{—}3010insT; CNTNAP1: NM_{—}003632:c.2993-2_{—}2994del, LGI4: NM_{—}139284:c.G793A, ADCY6: NM_{—}015270:c.C3346T, LMOD3: NM_{—}198271:c.135_{—}136insC and
instructions for use.

In one embodiment of the invention, the primer or probe may be labelled with a suitable marker. In another embodiment of the invention, the primer or probe may be coated on an array.

### Therapeutic Method

As previously mentioned the inventors demonstrate in the functional analysis of ADCY6 mutation in zebrafish model, severe and specific defects in peripheral myelin in spite of the presence of Schwann cells similar to those observed in AMC patients.

Accordingly a third object of the present invention is a nuclease for use in treating arthrogryposis multiplex congenita and/or congenital peripheral neuropathy (CPN) diseases and/or preventing progression of arthrogryposis multiplex congenita and/or congenital peripheral neuropathy diseases in a patient, wherein the presence of SNV at CNTNAP1, LGI4, ADCY6 and LMOD3 gene in a sample previously obtained from said patient, have been detected by a method of the invention previously described.

In particular embodiment the SNV is selected from the group consisting of CNTNAP1: NM_{—}003632:c.2901_{—}2902del, CNTNAP1: NM_{—}003632:c.3009_{—}3010insT; CNTNAP1: NM_{—}003632:c.2993-2_{—}2994del, LGI4:c.G793A, ADCY6:c.C3346T, LMOD3: c.135_{—}136insC and wherein
the presence of the allele (del) of CNTNAP1: NM_{—}003632:c.2901_{—}2902del is indicative of an arthrogryposis multiplex congenita and/or congenital peripheral neuropathy disease, and
the presence of the allele (insT) of CNTNAP1: NM_{—}003632:c.3009_{—}3010 insT is indicative of an arthrogryposis multiplex congenital and/or congenital peripheral neuropathy disease;
the presence of the allele (del) of CNTNAP1: NM_{—}003632:c.2993-2_{—}2994del is indicative of an arthrogryposis multiplex congenita and/or congenital peripheral neuropathy disease;
the presence of the allele (A) of LGI4: NM_{—}139284:c.G793A is indicative of an arthrogryposis multiplex congenita and/or congenital peripheral neuropathy disease;
the presence of the allele (T) of ADCY6: NM_{—}015270:c.C3346T is indicative of an arthrogryposis multiplex congenita and/or congenital peripheral neuropathy disease;
the presence of the allele (insC) of LMOD3: NM_{—}198271:c.135_{—}136 insC is indicative of an arthrogryposis multiplex congenita and/or congenital peripheral neuropathy disease.

A man skilled in the art, know as to design a specific nuclease in order to repair of genetic point mutations like SNVs located at CNTNAP1, LGI4, ADCY6 and LMOD3 gene, namely CNTNAP1: NM_{—}003632:c.2901_{—}2902del, CNTNAP1: NM_{—}003632:c.3009_{—}3010insT; CNTNAP1: NM_{—}003632:c.2993-2_{—}2994del, LGI4: NM_{—}139284:c.G793A, ADCY6: NM_{—}015270:c.C3346T, LMOD3: NM_{—}198271:c.135_{—}136insC.

The term “nuclease” or “endonuclease” means synthetic nucleases consisting of a DNA binding site, a linker, and a cleavage module derived from a restriction endonuclease which are used for gene targeting efforts. The synthetic nucleases according to the invention exhibit increased preference and specificity to bipartite or tripartite DNA target sites comprising DNA binding (i.e. TALE recognition site(s)) and restriction endonuclease target site while cleaving at off-target sites comprising only the restriction endonuclease target site is prevented.

Restriction endonucleases (also called restriction enzymes) as referred to herein in accordance with the present invention are capable of recognizing and cleaving a DNA molecule at a specific DNA cleavage site between predefined nucleotides. In contrast, some endonucleases such as for example Fokl comprise a cleavage domain that cleaves the DNA unspecifically at a certain position regardless of the nucleotides present at this position. Therefore, preferably the specific DNA cleavage site and the DNA recognition site of the restriction endonuclease are identical. Moreover, also preferably the cleavage domain of the chimeric nuclease is derived from a restriction endonuclease with reduced DNA binding and/or reduced catalytic activity when compared to the wildtype restriction endonuclease.

According to the knowledge that restriction endonucleases, particularly type II restriction endonucleases, bind as a homodimer to DNA regularly, the chimeric nucleases as referred to herein may be related to homodimerization of two restriction endonucleases subunits. Preferably, in accordance with the present invention the cleavage modules referred to herein have a reduced capability of forming homodimers in the absence of the DNA recognition site, thereby preventing unspecific DNA binding. Therefore, a functional homodimer is only formed upon recruitment of chimeric nucleases monomers to the specific DNA recognition sites. Preferably, the restriction endonuclease from which the cleavage module of the chimeric nuclease is derived is a type llP restriction endonuclease. The preferably palindromic DNA recognition sites of these restriction endonucleases consist of at least four or up to eight contiguous nucleotides. Preferably, the type llP restriction endonucleases cleave the DNA within the recognition site which occurs rather frequently in the genome, or immediately adjacent thereto, and have no or a reduced star activity. The type llP restriction endonucleases as referred to herein are preferably selected from the group consisting of: Pvull, EcoRV, BamHl, Bcnl, BfaSORF1835P, BfiI, Bgll, Bglll, BpuJl, Bse6341, BsoBl, BspD6I, BstYl, Cfr101, Ec118kl, EcoO1091, EcoRl, EcoRll, EcoRV, EcoR124l, EcoR124ll, HinPll, Hincll, Hindlll, Hpy99l, Hpy188l, Mspl, Munl, Mval, Nael, NgoMIV, Notl, OkrAl, Pabl, Pacl, PspGl, Sau3Al, Sdal, Sfil, SgrAl, Thal, VvuYORF266P, Ddel, Eco57l, Haelll, Hhall, Hindll, and Ndel.

Other nuclease for use in the present invention are disclosed in WO 2010/079430, WO2011072246, WO2013045480, Mussolino C, et al (Curr Opin Biotechnoi. 2012 October; 23(5):644-50) and Papaioannou I. et al (Expert Opinion on Biological Therapy, March 2012, Vol. 12, No. 3: 329-342) all of which are herein incorporated by reference.

Another object of the present invention is a method of treating arthrogryposis multiplex congenita and/or congenital peripheral neuropathy disease in a subject comprising the steps of:

a) providing a biological sample from a subject,

b) detecting in a biological sample obtained at step a) the presence or absence of an allelic variant of a single nucleotide variant (SNV) located in CNTNAP1, LGI4, ADCY6 and LMOD3 gene; and

if a SNVs is detected,

treating the subject with a nuclease through administration of a therapeutically effective amount of the nuclease.

In a particular embodiment, the SNV is selected from the group consisting of CNTNAP1: NM_{—}003632:c.2901_{—}2902del, CNTNAP1: NM_{—}003632:c.3009_{—}3010insT; CNTNAP1: NM_{—}003632:c.2993-2_{—}2994del, LGI4: NM_{—}139284:c.G793A, ADCY6: NM_{—}015270:c.C3346T, LMOD3: NM_{—}198271:c.135_{—}136insC and wherein
the presence of the allele (del) of CNTNAP1: NM_{—}003632:c.2901_{—}2902del is indicative of an arthrogryposis multiplex congenita and/or congenital peripheral neuropathy disease;
the presence of the allele (insT) of CNTNAP1: NM_{—}003632:c.3009_{—}3010insT is indicative of an arthrogryposis multiplex congenita and/or congenital peripheral neuropathy disease;
the presence of the allele (del) of CNTNAP1: NM_{—}003632:c.2993-2_{—}2994del is indicative of an arthrogryposis multiplex congenita and/or congenital peripheral neuropathy disease;
the presence of the allele (A) of LGI4: NM_{—}139284:c.G793A is indicative of an arthrogryposis multiplex congenita and/or congenital peripheral neuropathy disease;
the presence of the allele (T) of ADCY6: NM_{—}015270:c.C3346T is indicative of an arthrogryposis multiplex congenita and/or congenital peripheral neuropathy disease;
the presence of the allele (insC) of LMOD3: NM_{—}198271:c.135_{—}136 insC is indicative of an arthrogryposis multiplex congenita and/or congenital peripheral neuropathy disease.

The invention will be further illustrated by the following figures and examples. However, these examples and figures should not be interpreted in any way as limiting the scope of the present invention.

### FIGURES

FIG. 1—Percentage of AMC patients with associated symptoms and classified upon the primary tissue targets (Sk Mu: skeletal muscle, NMJ: neuromuscular junction or axoglial). * indicates a significant difference (P<0.02) between groups using Fisher's exact test. (P+D) AMC indicates that contractures involved both proximal and distal joints.

FIG. 2—Sanger sequencing of mutations identified in CNTNAP1, LGI4, ADCY6 and LMOD3 genes through whole exome sequencing in AMC families.

Arrows indicate mutant nucleotide positions. Open symbols: unaffected; filled symbols: affected; squares: male; circles: female. The nucleotide and amino acid changes are indicated as well as the genotype (homozygous) in affected individuals. Reverse strand is shown for the LMOD3 mutation in family K177.

FIG. 3—TEM analysis of nerve or skeletal muscle in AMC fetuses carrying deleterious mutations of CNTNAP1 (A, C), ADCY6 (E) and LMOD3 (G, H).

(A) Longitudinal section of sciatic nerve in AMC fetus A641 and control (B) of the same age gestational (32 w.g.). Note the marked widening of the node of Ranvier in AMC fetus. (C) Transverse ultrathin section showing the absence of large myelinated axons associated with thinner myelin sheaths in the AMC foetus K182 when compared to control (D) of the same gestational age (34 w.g.). (E) Lack of myelin surrounding axon (white arrow) while Schwann cell morphology appeared normal (black arrow) in AMC foetus A649 when compared to control of the same age (F, 40 w.g.). (G, H) TEM analysis of skeletal muscle in AMC patient K177. (G) Ultrastructural pattern of rods characteristic of nemalin myopathy. (H) In addition to rods, small dense rectangular structures (white arrow) are observed. Scale bars: A-B; E-F: 3.8 μm; C-D: 20 μm; G: 1 μm; H: 3.5 μm.

FIG. 4—Adcy6a and b genes are essential for PNS myelination. (A, B, E, F, I, J).

mbp mRNA expression by in situ hybridization in WT (A, B), embryos injected with the two control 5 mismatched Morpholinos (MOs) (0.3 pmole 5 mis adcy6a MO+0.3 pmole 5 mis adcy6b MO per embryo, E, F), and embryos injected with adcy6a and adcy6b MOs (0.3 pmole adcy6a MO+0.3 pmole adcy6b MO per embryo, I, J), respectively. For convenience, we used adcy6 MO instead of: adcy6a and b MOs. Note that mbp expression is severely reduced specifically in the PNS in embryos injected with both adcy6a and b MOs (compare I to A and E) while its expression is normal and comparable to controls in the CNS (compare J to B and F). Arrows in A and E indicate mbp expression along the Posterior Lateral Line nerve (PLLn). Arrows in B, F and J indicate the expression of mbp in the hindbrain and spinal cord while the arrowheads indicate its expression in the PLL ganglion (PLLg). Note the absence of mbp expression in the PLLg in J. (C, D, G, H, K, L) Schwann cells and PLLn labeling using the transgenic line foxd3::GFP and acetylated tubulin (ac Tub) antibody respectively. Note the presence of Schwann cells and PLLn in the adcy6 morphants as observed in controls (compare K to C and G; L to D and H). Arrows in C, G and K indicate Schwann cells along the PLLn and in D, H and L indicate the PLLn. Scale bar: 200 μm, A, B, E, F, I and J; 100 μm in C, D, G, H, K and L.

### Example

### Patients & Methods

Patients

We enrolled 65 affected fetuses, children, or adults belonging to 33 multiplex and/or consanguineous families with unexplained non-syndromic AMC from 2010 to 2013 in France. All cases were evaluated by obstetricians, fetopathologists, clinical geneticists, neonatologists or neuropediatricians. Any other defective organs were regarded as exclusion criteria. Targeted gene tests when performed did not lead to diagnosis. The parents of all patients provided written informed consents for genomic analysis of their children or fetuses and themselves in accordance with the ethical standards of our institutional review boards.

Genome-Wide Linkage Analysis

Whole genome SNP scanning was carried out according to Affymetrix 250K GeneChip Mapping Assay Manual. Multipoint linkage analysis and homozygosity mapping of SNP data were performed using the Alohomora⁷ and Merlin softwares.⁸

Whole Exome Sequencing (WES)

The Illumina TruSeq DNA Sample Prep kit v1 and the NimbleGen SeqCap EZ Human Exome Library v2.0 (targeting 44 Mb, from A631 to A648 DNA samples) or v3.0 (targeting 64 Mb, from A649 to K199 DNA samples) were used for library preparation and exome enrichment, respectively as previously described.⁹ Sequencing was performed on a Genome Analyzer IIx instrument using paired-end 75 bp reads and following the Illumina's protocol. Reads were aligned to the human reference genome sequence (UCSC hg19, NCBI build 37.3) via the BWA program.¹⁰ Variants were selected using the SAMtools¹¹ then annotated using Annovar softwares.¹² Reads with a coverage of at least 2× were filtered against dbSNPv131 database. Variants with a minor allele frequency (MAF) less <0.0015 and located in coding regions, intron-exon junctions or short coding insertions or deletions were selected. Then variants mapping to the candidate regions as determined by linkage analysis were selected. MAF was updated using dbSNPv132 database and EVS (ESP6500SI-V2). Integrative genomics viewer (IGV, v1.5.64)¹³ was used as visualization tool of WES variants. In silico prediction of the functional effect at the amino acid level was calculated using Polyphen-2 software (version 2.2.2).¹⁴

Real Time PCR Amplification of Genomic DNA, Reverse Transcription-PCR

Amplification and Sanger sequencing. They were conducted using primers as previously described.⁹ Sanger sequencing was used to validate variants fulfilling prioritization criteria and to analyze their segregation within each family. A cohort of 95 controls was analyzed for the selected variants when they were not known to be pathogenic so far.

Morphology of the Neuromuscular System

Muscle and nerve biopsy samples were processed as previously described.¹⁵ Nerve immunohistochemistry was carried out using phosphorylated neurofilament monoclonal antibody (1:200), myelin basic protein (MBP, 1:200) and 5100 protein polyclonal antibodies (1:2500, Dakopatts, Trappes). Muscle and nerve ultrastructural studies were carried out according to standardized protocols. Briefly, tissue samples were fixed in a 2% glutaraldehyde fixative solution, post-fixed with osmium tetroxide, and embedded in resin epoxy. Semi-thin sections were stained with toluidine blue. Ultra-thin sections were contrasted with uranyl acetate and lead citrate, and examined under a PHILIPS CM10 transmission electron microscope.

Knockdown of ADCY6 in Zebrafish

Embryos were cared for according to standard protocols.¹⁶ Foxd3::GFP transgenic line was used here.¹⁷ Antisense morpholino oligonucleotides (adcy6 MOs) were purchased from Gene Tools LLC and designed against the two corresponding orthologous zebrafish genes: adcy6a and adcy6b. The adcy6a MO was designed to target the 5′ UTR of adcy6a mRNA (XM_{—}001922714). The corresponding control “mismatch” morpholino had 5 nucleotides altered along its sequence. The same applies to adcy6b MO targeting adcy6b mRNA. 0.3 mM of each of the two morpholinos respectively designed against the two genes were mixed (either a mix of the 5′UTR MOs or the controls ones; final concentration: 0.6 mM). 1 nl of morpholino was injected into 1-4 cell stage embryos as previously described.18 Embryos were fixed in 4% paraformaldehyde and stained as whole mounts following standard in situ hybridization protocols and using mbp (myelin basic protein) probe. For immunostaining using anti-acetylated tubulin antibody (Sigma, 1:1000), embryos were fixed in 4% paraformaldehyde and stained as whole mounts. Image acquisition was performed using a Zeiss confocal microscope.

Results

Phenotypic Characterization of AMC Patients

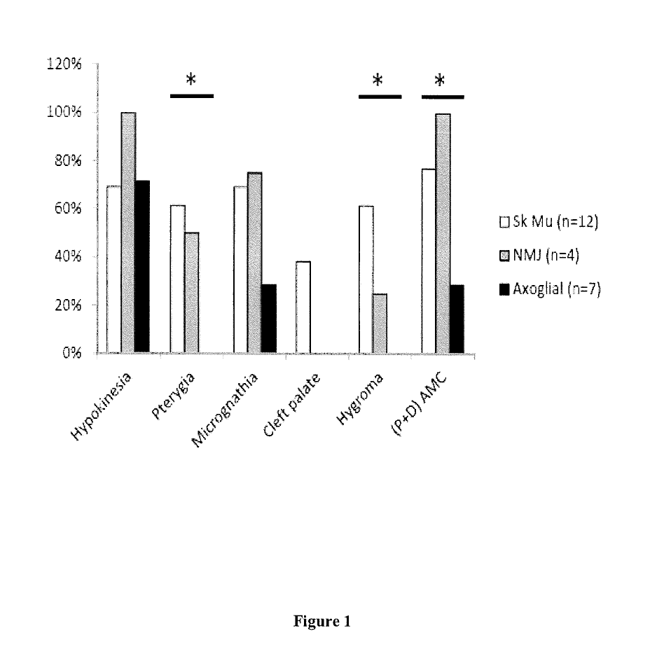

The main criteria were the gestational age of AMC diagnosis based on ultrasound examination (weeks' gestation, w.g., the earliest one in multiplex families), the topography of joint contractures, the amount of amniotic fluid and associated symptoms including reduced fetal mobility, pterygium, micrognathia, cleft palate, cystic hygroma and the evolution of pregnancies. Based on ultrasound examination, the age of AMC diagnostic showed a significant variability, from 11 to 32 w.g. indicating a marked heterogeneity of fetal onset. Reduced fetal mobility was associated with AMC in 25 out of 29 affected individuals (86%). No information was available in 2 families. The primary defects were established based on gene identification and function (see below) and were classified as skeletal muscle, neuromuscular junction or axoglial AMC. The distribution of associated symptoms was compared between classes using Fisher's exact test. AMC associated with pterygia, hygroma and both distal and proximal contractures are significantly more frequent in skeletal muscle class (P<0.02, FIG. 1).

Identification of Mutations in Genes Known to be Responsible for Arthrogryposis Multiplex Congenita or Neuromuscular Disorders

Candidate loci were identified in each family by multipoint linkage analysis combined with homozygosity mapping in the consanguineous ones. When a single gene known to be involved in AMC was identified in candidate disease loci, Sanger sequencing of exons and intron-exon junctions was performed. This was successfully applied in 3 families out of 31 (10%) including a family linked to mutations of RYR1 (family B415), TRPV4 (family B651, Table 1) or ECEL1 (family B192, 5) When numerous or none AMC genes were identified in candidate loci, whole exome sequencing was performed using the DNA sample of one affected individual per family (n=28). The median coverage of the WES was 33 to 138 (average: 74). Variants mapping to the disease loci were selected reducing the number of variants to 1-32%.

As a first filtering, homozygous or compound heterozygous variants were selected in autosomal recessive forms and heterozygous variants in autosomal dominant ones. Variants were then filtered against a list of genes known to be involved in AMC or neuromuscular disorders (NMD). Using this 1st filter, mutations in NEB, RYR1, SYNE1, TNNT3, TTN or CHRNG genes were successfully identified in 10 out of 33 families (30%, Table 1). Interestingly, homozygous stop gained mutation was found in exon 136 of the SYNE-1 gene in family K168. In family A642, a non-sense mutation on one allele and a frameshift mutation on the other allele of the TTN gene were found. In a consanguineous family (A659), a homozygous splice mutation was found in RBBP8 gene (Table 1). Mutations of this gene have been reported in Seckel syndrome type 2 which may associate microcephaly, holoprosencephaly and arthrogryposis¹⁸ suggesting that the affected foetus had a syndromic AMC not detected during pregnancy.

In 17 families, allelic mutations were not identified using the above criteria. In 4 autosomal recessive families, a single heterozygous variant in AMC or NMD genes and mapping to candidate loci was identified. Non-covered exons visualized by the IGV software were then sequenced by Sanger method. Allelic mutations were found in ECEL1, RAPSN, RYR1 and NEB (Table 1). When no mutations were identified using these filters, the hypothesis of dominant de novo and recurrent mutation mimicking autosomal recessive inheritance was tested by selecting variants within all known AMC or NMD genes without any linkage data filter. A single missense mutation of the MYH3 gene was identified in the two affected fetuses of family A640 with a paternal somatic mosaicism (Table 1).

Identification of New Genes in AMC

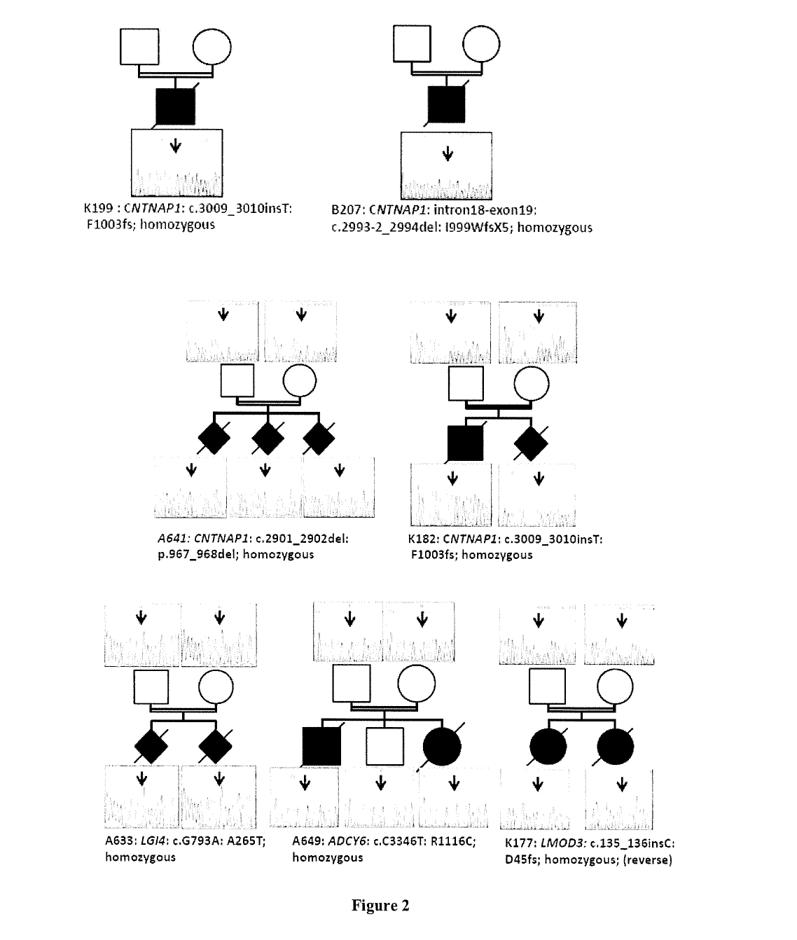

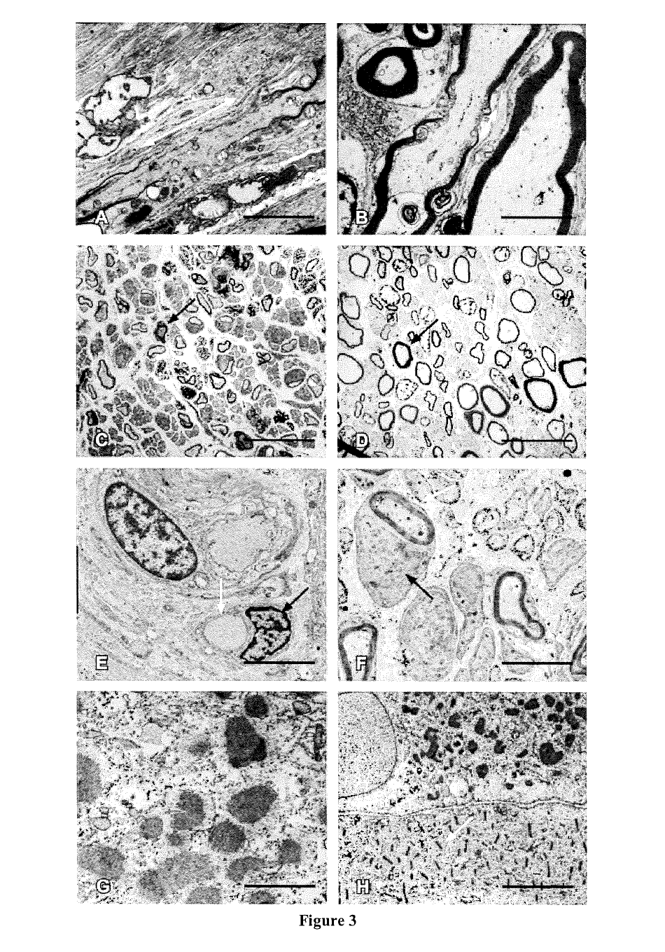

Finally, four new genes have been identified in 5 families. This was based on genetic criteria (FIG. 1), the morphological analysis of the neuromuscular system and the availability or the generation of animal models. In two consanguineous families (K182 and A641), distinct homozygous frameshift mutations were found in CNTNAP1 encoding Caspr (Table 1, FIG. 2). In family A641, the three fetuses born from consanguineous parents carry an homozygous 1 bp deletion in CNTNAP1 exon 18 (c.2901_{—}2902del) leading to frameshift and premature stop codon (P967PfsX12). In family K182, affected patients carry an homozygous 1 bp insertion in CNTNAP1 exon 19 (c.3009_{—}3010 insT) leading to frameshift (F1003fs). Two additional families with similar phenotype (see below) were found to carry deleterious mutations in the same gene using either the combination of both linkage analysis with WES (family K199) or homozygosity mapping only (B207). In the consanguineous family K199, affected patients carry the same homozygous 1 bp insertion in CNTNAP1 exon 19 (c.3009_{—}3010 insT) as the unrelated family K182. In family B207, an homozygous frameshift mutation in exon 19 of CNTNAP1 was found in the patient (intron18-exon19:c.2993-2_{—}2994del, I999WfsX5). In families K182 and A641, both parents were heterozygous for the mutation as expected. In families K199 and B207, DNA samples from parents were not available. These mutations were absent in 95 ethnically matched controls. The c.2901_{—}2902del mutation was found at a very low minor allele frequency (MAF: 0.00016) in the current Exome Variant Server database (EVS, ESP6500SI-V2). The other mutations were found in neither EVS nor dbSNPv138.

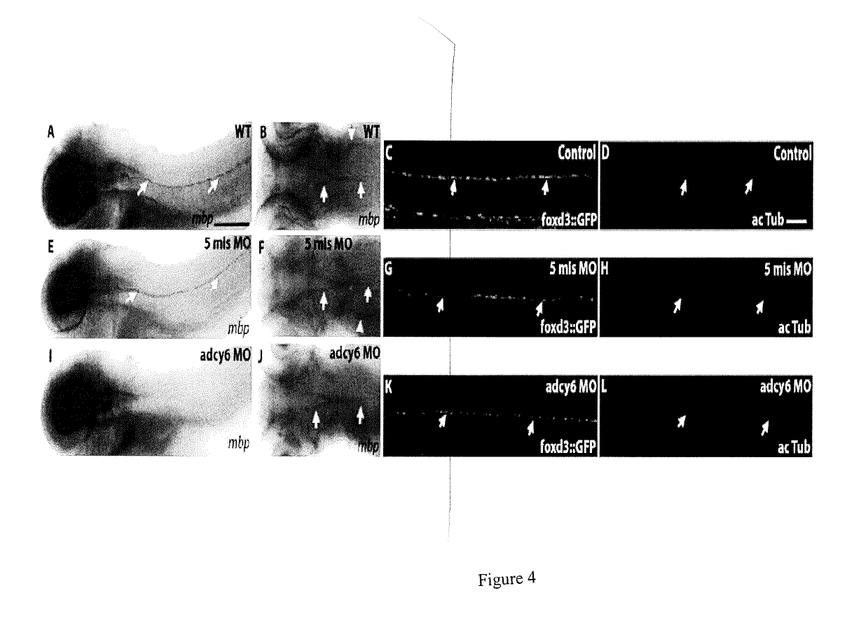

In the four families, the fetal phenotype was quite similar and characterized by a late onset during pregnancy (from 28 w.g.), polyhydramnios and distal joint contractures including talipes equinovarus and both proximal and distal interphalangeal joint contractures of the hands. Proximal joints were not involved. At birth, the patients displayed severe hypotonia, facial diplegia and a lack of swallowing, autonomous respiratory function and deep tendon reflexes. Motor nerve conduction velocity was markedly reduced (<10 m/sec). In all patients, death occurred within the first two months of life. Since CASPR is known to play a key role in the delineation of the axonal domains of myelinated axons in mice (19), Transmission Electron Microscopy (TEM) of sciatic nerve in the A641 and K182 index cases was performed and revealed two major abnormalities (FIG. 3). First, examination of longitudinal sections revealed a marked widening of the node of Ranvier in patient A641 (3.98+/−0.56; n=5) when compared to control (1.12+/−0.98; n=4, P<0.05; FIG. 3, A, B). Second, on transverse sections, myelinated axons of patient nerve (n=50) display significant reduced surface associated with thinner myelin sheath when compared to an age-matched control case (n=69, FIG. 3, C, D, P=0.04 and P<0.0003, respectively, Kruskal-Wallis test). In another consanguineous family (A633), a homozygous missense mutation was found at the last nucleotide of exon 7 of LGI4 (Table 1, FIG. 2), leading to intron 7 retention, frameshift and premature stop codon. Nerve sample was not available from the affected fetus.

In the consanguineous family A649, a homozygous missense mutation predicted to be damaging with a high score (1.00) was found in ADCY6 (Table 1, FIG. 2). Nerve immunolabelling using S100 protein antibody revealed a normal number of Schwann cells but all nerve fascicles were negative for MBP antibodies. TEM of nerve revealed no myelinated axons (FIG. 3). Some redundant basal lamina of Schwann cells were also observed, suggesting inability of Schwann cells to properly myelinate axons. Moreover, a knockdown of the ADCY6 orthologs in zebrafish led to a very similar phenotype with a loss of mbp expression in the PNS (showed here is the Posterior Lateral Line nerve, n=17/17) while the central nervous system (CNS) mbp expression is comparable to controls (FIG. 4). Furthermore, we also found no defects in Schwann cell migration and axonal growth in the morphants (FIG. 4).

In one consanguineous family (K177), a homozygous frameshift mutation was found in LMOD3 (Table 1, FIG. 2). Modified Gomori trichrome staining of the muscle biopsy revealed the presence of multiple rods consistent with the diagnosis of nemalin myopathy. TEM confirmed the presence of rods with their characteristic lattice pattern associated with small dense rectangular structures suggesting Z bands disruption (FIG. 3).

In the remaining 7 families (A658, K180, K171, K169, K174, K165 and A657), no mutations were found, whatever filter used suggesting either the lack of coverage of allelic mutations in exonic or intronic regions or non genetic causes of AMC.

### DISCUSSION

Our study is the first large cohort of undiagnosed AMC individuals investigated by combining genetic mapping with whole exome sequencing. Our results confirm the clinical and genetic heterogeneity of non-syndromic AMC. We detected pathogenic mutations in known AMC or NMD genes in 19 families (61%) and new genes in 7 families (21%). Among known disease genes, our study confirmed that mutations of SYNE-1 are responsible for AMC20 and revealed an AMC family carrying allelic non-sense and frameshift mutations of the TTN gene extending the clinical spectrum of TTN gene mutations.

In 7 families, 4 novel genes were identified and include CNTNAP1, LGI4, ADCY6 and LMOD3. A causative role of mutations in the CNTNAP1 gene encoding CASPR was established by the identification of distinct homozygous frameshift mutations in four unrelated families. In addition, dramatic reduction of motor nerve conduction velocities associated with severe abnormalities of the nodes of Ranvier and myelinated axons were observed. The node of Ranvier, the flanking paranodal junctions, and the juxtaparanodes underlie saltatory conduction of action potentials along myelinated axons, an essential process for neuronal function. The paranodal junctions consist of a complex of the axonal proteins Caspr¹⁹ and contactin²⁰ and the glial isoform of neurofascin.²¹ Mice that lack Caspr exhibit significant motor paresis associated with a defect in the formation of paranodal junctions indicating that Caspr plays a key role in this process.²² Interestingly, in another family, a homozygous splice mutation leading to premature stop codon has been identified in LGI4. The claw paw mutation, a spontaneous mutation of LGI4 in mouse, is responsible for forelimb deformity and delayed myelination throughout the PNS associated with dramatically shortened internodal distances.²³⁻²⁵ Lgi4 interacts with ADAM22, a component of the potassium voltage-gated channel complex that recruits membrane-associated guanylate kinases to juxtaparanodes of myelinated axons.²⁶ Both Caspr and LGI4 have an essential role in the formation or the maintenance of Ranvier domains and were found to be mutated in human AMC.

A homozygous missense mutation in ADCY6 has been found in a consanguineous family. Ultrastructural morphology of patient nerve sample revealed the presence of Schwann cells but the lack of myelin in the PNS. Although mutation of this gene has been found in a single family, knock down of the orthologous genes in zebrafish provided evidence for major myelin defects in the PNS, data similar to those found in patients. These results strongly support an essential and so far unknown role of ADCY6 in this process. ADCY6 encodes a protein that belongs to adenylate cyclase family responsible for the synthesis of cAMP (27). During development, promyelinating Schwann cells associate with one segment of an axon and differentiate into myelinating Schwann cells to form the myelin sheath. Elevation of cAMP can mimic axonal contact in vitro and is thought to upregulate myelinating signals (28-30). The G protein-coupled receptor Gpr126 has been recently shown to be required in Schwann cells for myelination (31). Elevation of cAMP in gpr126 zebrafish mutants could restore myelination suggesting that Gpr126 drives the differentiation of promyelinating Schwann cells by elevating cAMP levels (31). Our data suggest that ADCY6 is involved in the same Gpr126-cAMP pathway for myelination of Schwann cells.

Finally, mutation of LMOD3 has been identified as a possible causative gene in nemalin myopathy found in K177 family. LMOD3 is one of three paralogous genes encoding leimodins involved in the assembly of actin cytoskeleton,³² data consistent with the pathogenic mechanism frequently involved in nemalin myopathy, a disorder of sarcomeric thin filaments.³³ Additional patients carrying mutations of the same gene should be found and functional studies should be done to conclude LMOD3 as the causative disease gene.

In conclusion, this strategy allowed the identification of new axoglial human diseases characterized by primary defect of nodes of Ranvier and the so far unknown link of the ADCY6 protein with PNS myelination. These genes may be regarded as strong candidates in undiagnosed congenital peripheral neuropathies. In spite of several pitfalls identified mostly due to the lack of coverage of either some exons or large genomic rearrangements, parallel next generation sequencing (NGS) of AMC genes, and novel ones identified in this study should led to a diagnosis in more than 75% of cases, since our study concentrated on undiagnosed AMC only, without previous integration of muscle or nerve morphological studies. Such targeted NGS approach should avoid the identification of clinically relevant mutations not related to AMC. Establishing early diagnosis should provide accurate information on the prognosis.

TABLE 1
Identified mutations in genes associated with AMC

| | Inher- | | | MAF | MAF | | | | |
|---|---|---|---|---|---|---|---|---|---|
| Code | itance | Gene | Transcript Ref.: Nucleotide change | (dbSNP) | (EVS) | Genotype | Type | Protein change | Target |
| A641 | AR* | CNTNAP1 | NM_003632: exon18: | 0 | 0.00016 | homozyg. | frameshift | P967PfsX12 | Axoglial |
| | | | c.2901_2902del | | | | | | |
| K182 | AR* | CNTNAP1 | NM_003632: exon19: | 0 | 0 | homozyg. | frameshift | F1003fs | Axoglial |
| | | | c.3009_3010insT | | | | | | |
| K199 | AR* | CNTNAP1 | NM_003632: exon19: | 0 | 0 | homozyg. | frameshift | F1003fs | Axoglial |
| | | | c.3009_3010insT | | | | | | |
| B207 | AR* | CNTNAP1 | NM_003632: intron18-exon19: | 0 | 0 | homozyg. | frameshift | I999WfsX5 | Axoglial |
| | | | c.2993-2_2994del | | | | | | |
| A649 | AR* | ADCY6 | NM_015270: exon20: c.C3346T | 0 | 0 | homozyg. | missense (d; 1) | R1116C | Axoglial |
| A633 | AR* | LG14 | NM_139284: exon7: c.G793A | 0 | 0 | homozyg. | missense | A265T | Axoglial |
| | | | | | | | (d; 0.96) | | |
| A635 | AR | ECEL1 | NM_004826: exon4: c.925delA | 0 | 0 | comp. het. | frameshift | K309fs | Axoglial |
| A635 | AR | ECEL1 | NM_004826: exon2: c.C33G | 0 | 0 | comp. het. | stop gained | Y11X | Axoglial |
| B192 | AR* | ECEL1 | NM_004826: c.1685 + 1G > T | 0 | 0 | homozyg. | splice | K552AfsX33 | Axoglial |
| B651 | AD | TRPV4 | NM_021625: exon6: c.G947A | 0 | 0 | heterozyg. | missense (p) | R316H | Axoglial |
| A638 | AR* | CHRNG | NM_005199: exon2: c.117_118insC | 0 | 0 | homozyg. | frameshift | P39fs | NMJ |
| A651 | AR* | CHRNG | NM_005199: exon7: c.C715T | 0 | 0 | homozyg. | missense (p) | R239C | NMJ |
| A662 | AR* | CHRNG | NM_005199: exon7: c.C715T | 0 | 0 | homozyg. | missense (p) | R239C | NMJ |
| A650 | AR | RAPSN | NM_032645: exon2: c.C264A | 0.0014 | 0.001 | comp. het. | missense (p) | N88K | NMJ |
| A650 | AR | RAPSN | NM_032645: EX1_2del | 0 | 0 | comp. het. | deletion | Ex1_2del | NMJ |
| A640 | AD | MYH3 | NM_002470: exon28: c.T3959C | 0 | 0 | heterozyg. | missense | L1320P | Sk Mu |
| | | | | | | | (d; 0.99) | | |
| A646 | AR* | NEB | NM_001164508: exon41: | 0 | 0 | homozyg. | frameshift | A1620fs | Sk Mu |
| | | | c.4858_4866del | | | | | | |
| A631 | AR* | NEB | NM_001164508: c.9832 − 1G > A | 0 | 0 | homozyg. | splice | Y3278MfsX22 | Sk Mu |
| B415 | AR | RYR1 | NM_000540: EX70_71del | 0 | 0 | comp. het. | deletion | EX70_71del | Sk Mu |
| B415 | AR | RYR1 | NM_000540: exon8: c.G644A | 0 | 0 | comp. het. | missense (d; 1) | G215E | Sk Mu |
| A648 | AR | RYR1 | NM_000540: exon46: c.G7373A | 0 | 0 | comp. het. | missense (p) | R2458H | Sk Mu |
| A648 | AR | RYR1 | NM_000540: c.14364 + 1G > A | 0 | 0 | comp. het. | splice | W4768CfsX11 | Sk Mu |
| A636 | AR | RYR1 | NM_000540: exon46: c.G7373A | 0 | 0 | comp. het. | missense (p) | R2458H | Sk Mu |
| A636 | AR | RYR1 | NM_000540: exon90: c.T12580C | 0 | 0 | comp. het. | missense | F4194L | Sk Mu |
| | | | | | | | (d; 0.95) | | |
| A656 | AR* | RYR1 | NM_000540: exon26: c.G3449A | 0 | 0 | homozyg. | missense (d; 1) | C1150Y | Sk Mu |
| K166 | AR* | RYR1 | NM_000540: exon57: c.C8758T | 0 | 0 | homozyg. | stop gained | R2920X | Sk Mu |
| K168 | AR* | SYNE1 | NM_182961: exon136: c.C24577T | 0 | 0 | homozyg. | stop gained | R8193X | Sk Mu |
| A663 | AD | TNNT3 | NM_006757: exon10: c.G188A | 0 | 0 | heterozyg. | missense (p) | R63H | Sk Mu |
| A642 | AR | TTN | NM_133378: exon195: | 0 | 0 | comp. het. | frameshift | Y12621_V12622 | Sk Mu |
| | | | c.37862_37863insA | | | | | delinsX | |
| A642 | AR | TTN | NM_133378: exon307: c.C96388T | 0 | 0 | comp. het. | stop gained | R32130X | Sk Mu |
| K177 | AR* | LMOD3 | NM_198271: exon1: c.135_136insC | 0 | 0 | homozyg. | frameshift | D45fs | Sk Mu |
| A659 | AR* | RBBP8 | NM_002894: c.2455 − 4T > G | 0 | 0.00008 | homozyg. | splice | Y819VfsX33 | Other |

AR: autosomal recessive;
AD: autosomal dominant;
*consanguinity;
Transcript Ref.: Transcript Reference;
homozyg.: homozygous;
comp. het.: compound heterozygous;
heterozyg.: heterozygous;
Type: (p) = known pathogenic mutation, (d) = damaging mutation as determined by Polyphen-2 prediction with score;
NMJ: neuromuscular junction;
Sk Mu: skeletal muscle.
Mutations were confirmed by Sanger sequencing or real time PCR and splice mutations by RNA analysis.

### REFERENCES

Throughout this application, various references describe the state of the art to which this invention pertains. The disclosures of these references are hereby incorporated by reference into the present disclosure.
1. Hall J G. Genetic aspects of arthrogryposis. Clin Orthop 1985; 194:44-53.
2. Fahy M J, Hall J G. A retrospective study of pregnancy complications among 828 cases of arthrogryposis. Genet Couns 1990; 1:3-11.
3. Bamshad M, Van Heest A E, Pleasure D. Arthrogryposis: a review and update. J Bone Joint Surg Am 2009; 91: Suppl 4:40-6.
4. Navti O B, Kinning E, Vasudevan P, et al. Review of perinatal management of arthrogryposis at a large UK teaching hospital serving a multiethnic population. Prenat Diagn 2010; 30:49-56.
5. Dieterich K, Quijano-Roy S, Monnier N, et al. The neuronal endopeptidase ECEL1 is associated with a distinct form of recessive distal arthrogryposis. Hum Mol Genet 2013 22:1483-92.
6. McMillin M J, Below J E, Shively K M, et al. Mutations in ECEL1 cause distal arthrogryposis type 5D. Am J Hum Genet 2013; 92:150-6
7. Ruschendorf F and Nurnberg P. ALOHOMORA: a tool for linkage analysis using 10K SNP array data. Bioinformatics 2005; 21:2123-2125.
8. Abecasis G R, Cherny S S, Cookson W O, Cardon L R. Merlin-rapid analysis of dense genetic maps using sparse gene flow trees. Nat Genet 2002; 30:97-101.
9. Zhou J, Tawk M, Tiziano F D, et al. Spinal muscular atrophy associated with progressive myoclonic epilepsy is caused by mutations in ASAH1. Am J Hum Genet 2012; 91:5-14
10. Li H, Durbin, R. Fast and accurate short read alignment with Burrows-Wheeler transform. Bioinformatics 2009; 25:1754-1760 (a)
11. Li H, Handsaker B, Wysoker A, et al. 1000 Genome Project Data Processing Subgroup. The Sequence Alignment/Map format and SAMtools. Bioinformatics 2009; 25:2078-9 (b).
12. Wang K, Li M, Hakonarson H. ANNOVAR: functional annotation of genetic variants from high-throughput sequencing data. Nucleic Acids Res 2010; 38:e164.
13. Robinson J T, Thorvaldsdóttir H, Winckler W, et al. Integrative Genomics Viewer. Nature Biotechnology 2011; 29:24-26
14. Adzhubei I A, Schmidt S, Peshkin L, et al. A method and server for predicting damaging missense mutations. Nat Methods 2010; 7:248-249.
15. Dubowitz V, Sewry C A, Fitzsimons R. Muscle biopsy: a practical approach. 2nd ed. London. Philadelphia: Baillière Tindall, 1985.
16. Westerfield M. The zebrafish book. A guide for the laboratory use of zebrafish (Danio rerio). EugeneOR: University of Oregon Press. 1995.
17. Gilmour D T, Maischein H M, Nüsslein-Volhard C. Migration and function of a glial subtype in the vertebrate peripheral nervous system. Neuron 2002; 34:577-88.
18. Sarici D, Akin M A, Kara A, Doganay S, Kurtoglu S. Seckel syndrome accompanied by semilobar holoprosencephaly and arthrogryposis. Pediatr Neurol 2012; 46:189-91.
19. Peles E, Nativ M, Lustig M, et al. Identification of a novel contactin-associated transmembrane receptor with multiple domains implicated in protein-protein interactions. EMBO J 1997; 16:978-88
20. Rios J C, Melendez-Vasquez C V, Einheber S, et al. Contactin-associated protein (Caspr) and contactin form a complex that is targeted to the paranodal junctions during myelination. J Neurosci 2000; 20:8354-64
21. Tait S, Gunn-Moore F, Collinson J M, et al. An oligodendrocyte cell adhesion molecule at the site of assembly of the paranodal axo-glial junction. J Cell Biol 2000; 150:657-66
22. Bhat M A, Rios J C, Lu Y, et al. Axon-glia interactions and the domain organization of myelinated axons requires neurexin IV/Caspr/Paranodin. Neuron 2001; 30:369-383
23. Bermingham J R Jr, Shearin H, Pennington J, et al. The claw paw mutation reveals a role for Lgi4 in peripheral nerve development. Nat Neurosci 2006; 9:76-84
24. Henry E W, Eicher E M. & Sidman R L. The mouse mutation claw paw: forelimb deformity and delayed myelination throughout the peripheral nervous system. J Hered 1991; 82:287-294
25. Koszowski A G, Owens G C & Levinson S R. The effect of the mouse mutation claw paw on myelination and nodal frequency in sciatic nerves. J Neurosci 1998; 18:5859-5868
26. Ogawa Y, Oses-Prieto J, Kim M Y, et al. ADAM22, a Kv1 channel-interacting protein, recruits membrane-associated guanylate kinases to juxtaparanodes of myelinated axons. J Neurosci 2010; 30:1038-48
27. Edelhoff S, Villacres E C, Storm D R, Disteche C M. Mapping of adenylyl cyclase genes type I, II, III, IV, V, and VI in mouse. Mamm Genome 1995; 6:111-3.
28. Morgan, L., Jessen, K. R. and Mirsky, R. (1991) The effects of cAMP on differentiation of cultured Schwann cells: progression from an early phenotype (04+) to a myelin phenotype (P0+, GFAP-, N-CAM-, NGF-receptor-) depends on growth inhibition. J. Cell Biol., 112, 457-467.
29. Monuki, E. S., Weinmaster, G., Kuhn, R. and Lemke, G. (1989) SCIP: a glial POU domain gene regulated by cyclic AMP. Neuron, 3, 783-793.
30. Scherer, S. S., Wang, D. Y., Kuhn, R., Lemke, G., Wrabetz, L. and Kamholz, J. (1994) Axons regulate Schwann cell expression of the POU transcription factor SCIP. J. Neurosci., 14, 1930-1942.
31. Monk, K. R., Naylor, S. G., Glenn, T. D., Mercurio, S., Perlin, J. R., Dominguez, C., Moens, C. B. and Talbot, W. S. (2009) A G protein-coupled receptor is essential for Schwann cells to initiate myelination. Science, 325, 1402-1405.
32. Nanda V, Miano J M. Leiomodin 1, a new serum response factor-dependent target gene expressed preferentially in differentiated smooth muscle cells. J Biol Chem 2012; 287:2459-67.
33. Sanoudou D, Beggs A H. Clinical and genetic heterogeneity in nemaline myopathy—a disease of skeletal muscle thin filaments. Trends Mol Med 2001; 7:362-8

TABLE 2
Useful nucleotide (primers) for practicing the invention

| | | | | | pcr |
|---|---|---|---|---|---|
| | | | | | product |
| gene | primer | Sequence | size | Tm | size |
| LGI4 | LGI-Ex1F | aagacatacgaacagggaggaa(SEQ ID NO: 1) | 22 | 60.00 | 528 |
| LGI4 | LGI-Ex1R | cttctgctccttcccagatg (SEQ ID NO: 2) | 20 | 59.94 | |
| LGI4 | LGI-Ex2F | gaggagggttgagtgctgtc (SEQ ID NO: 3) | 20 | 59.84 | 492 |
| LGI4 | LGI-Ex2R | agcccccatctgacattttaac (SEQ ID NO: 4) | 22 | 61.4 | |
| LGI4 | LGI-Ex3F | gggagtgtatgctcacacagat (SEQ ID NO: 5) | 22 | 59.09 | 461 |
| LGI4 | LGI-Ex3R | ttcatcagtatcacccaaagca (SEQ ID NO: 6) | 22 | 60.5 | |
| LGI4 | LGI-Ex4F | cacagtgaaactggcaatgact (SEQ ID NO: 7) | 22 | 60.22 | 471 |
| LGI4 | LGI-Ex4R | tcacacatgagtaagggtgtcc (SEQ ID NO: 8) | 22 | 59.89 | |
| LGI4 | LGI-Ex5F | gcttcatcgaggacaatgagat (SEQ ID NO: 9) | 22 | 60.61 | 423 |
| LGI4 | LGI-Ex5R | cccacctcaggcaagtgt (SEQ ID NO: 10) | 18 | 59.66 | |
| LGI4 | LGI-Ex6F | gtgcccacttctggttgg (SEQ ID NO: 11) | 18 | 60.09 | 535 |
| LGI4 | LGI-Ex6R | cccatggcactcctatatcact (SEQ ID NO: 12) | 22 | 60.23 | |
| LGI4 | LGI-Ex7F | caaacatctacagatcaagattcca (SEQ ID NO: 13) | 25 | 59.55 | 580 |
| LGI4 | LGI-Ex7R | cccttccagccacaggag (SEQ ID NO: 14) | 18 | 61.80 | |
| LGI4 | LGI-Ex8aF | gtgtcctgcaagccactggt (SEQ ID NO: 15) | 20 | 63.67 | 482 |
| LGI4 | LGI-Ex8aR | atgtagcgtgtgaggcacag (SEQ ID NO: 16) | 20 | 59.93 | |
| LGI4 | LGI-Ex8bF | ggcttttacccgcaccagag (SEQ ID NO: 17) | 20 | 64.14 | 472 |
| LGI4 | LGI-Ex8bR | atttgggggtagaatcaggatg (SEQ ID NO: 18) | 22 | 61.25 | |
| LGI4 | LGI-Ex9aF | aaggtgaaactgacagagtcca (SEQ ID NO: 19) | 22 | 58.84 | 495 |
| LGI4 | LGI-Ex9aR | ccttaaagcaagcagcaaagag (SEQ ID NO: 20) | 22 | 60.65 | |
| LGI4 | LGI-Ex9bF | gtcttccagccactgctcat (SEQ ID NO: 21) | 20 | 60.42 | 446 |
| LGI4 | LGI-Ex9bR | agtaggctgggaccctctatgt (SEQ ID NO: 22) | 22 | 60.39 | |
| LMOD3 | LMODEX1-F | aaatatctcaagggtgtgttaacttg (SEQ ID NO: 23) | 26 | 59.01 | 624 |
| LMOD3 | LMODEX1-R | cccaattttccccttgtaaat (SEQ ID NO: 24) | 21 | 59.09 | |
| LMOD3 | LMODEX2A-F | aactgcttctgaaattaaacacca (SEQ ID NO: 25) | 24 | 59.27 | 585 |
| LMOD3 | LMODEX2A-R | cactttgttcttgggcctct (SEQ ID NO: 26) | 20 | 59.33 | |
| LMOD3 | LMODEX2B-F | tgatgatgacgacgaaggag (SEQ ID NO: 27) | 20 | 59.79 | 599 |
| LMOD3 | LMODEX2B-R | ccaacatgtgcctctgattg (SEQ ID NO: 28) | 20 | 60.11 | |
| LMOD3 | LMODEX2C-F | caatgtgggtgcagatgaga (SEQ ID NO: 29) | 20 | 60.69 | 551 |
| LMOD3 | LMODEX2C-R | gcgatggctttttcatcatt (SEQ ID NO: 30) | 20 | 60.05 | |
| LMOD3 | LMODEX2D-F | gaggacccaagccagattc (SEQ ID NO: 31) | 19 | 59.60 | 543 |
| LMOD3 | LMODEX2D-R | gagatggggtgctacaggtg (SEQ ID NO: 32) | 20 | 60.53 | |
| ADCY6 | ADCY-ex1aF | ggggatacccttgttgccta (SEQ ID NO: 33) | 20 | 61.06 | 466 |
| ADCY6 | ADCY-ex1aR | gtcacctcggtatcctcgaa (SEQ ID NO: 34) | 20 | 60.07 | |
| ADCY6 | ADCY-ex1bF | gtggatgaacggaaaacagc (SEQ ID NO: 35) | 20 | 60.50 | 477 |
| ADCY6 | ADCY-ex1bR | gtggaaagccagcagcac (SEQ ID NO: 36) | 18 | 60.57 | |
| ADCY6 | ADCY-ex1cF | ttcgaggataccgaggtgac (SEQ ID NO: 37) | 20 | 60.07 | 484 |
| ADCY6 | ADCY-ex1cR | aggagcgtgtaggcgatgta (SEQ ID NO: 38) | 20 | 60.82 | |
| ADCY6 | ADCY-ex1dF | gtggcactgttggcctgt (SEQ ID NO: 39) | 18 | 60.76 | 500 |
| ADCY6 | ADCY-ex1dR | agacccctcaaccctagcc (SEQ ID NO: 40) | 19 | 60.46 | |
| ADCY6 | ADCY-ex2F | cctgtgggtaatgggagaga (SEQ ID NO: 41) | 20 | 59.92 | 493 |
| ADCY6 | ADCY-ex2R | tccttcccttggacaggac (SEQ ID NO: 42) | 19 | 60.03 | |
| ADCY6 | ADCY-ex3F | tcaatggagggtggtcaga (SEQ ID NO: 43) | 19 | 60.03 | 478 |
| ADCY6 | ADCY-ex3R | caaagagctcattcagggtca (SEQ ID NO: 44) | 21 | 60.38 | |
| ADCY6 | ADCY-ex4F | ttgccatggagatgaaagaa (SEQ ID NO: 45) | 20 | 59.20 | 470 |
| ADCY6 | ADCY-ex4R | tgaatgggaaggaattggag (SEQ ID NO: 46) | 20 | 59.86 | |
| ADCY6 | ADCY-ex5F | gagctctttgcccggttt (SEQ ID NO: 47) | 18 | 60.34 | 590 |
| ADCY6 | ADCY-ex5R | cagatggcattccctctagc (SEQ ID NO: 48) | 20 | 59.80 | |
| ADCY6 | ADCY-ex6F | tgctgttgaacacagcctta (SEQ ID NO: 49) | 20 | 58.06 | 541 |
| ADCY6 | ADCY-ex6R | aatgtgctgctccttgaggt (SEQ ID NO: 50) | 20 | 59.87 | |
| ADCY6 | ADCY-ex7F | aggctggtacgtgaggtgac (SEQ ID NO: 51) | 20 | 60.18 | 562 |
| ADCY6 | ADCY-ex7R | atcagcccttccatggagtt (SEQ ID NO: 52) | 20 | 60.85 | |
| ADCY6 | ADCY-ex8F | gtacctcaaggagcagcaca (SEQ ID NO: 53) | 20 | 59.04 | 494 |
| ADCY6 | ADCY-ex8R | caacactgtgctgagggtgt (SEQ ID NO: 54) | 20 | 59.78 | |
| ADCY6 | ADCY-ex9F | tgtgactgccatttccatgt (SEQ ID NO: 55) | 20 | 59.97 | 393 |
| ADCY6 | ADCY-ex9R | ggccatgtgagaaacaatga (SEQ ID NO: 56) | 20 | 59.50 | |
| ADCY6 | ADCY-ex10F | gcattgatgattccagcaaa (SEQ ID NO: 57) | 20 | 59.63 | 576 |
| ADCY6 | ADCY-ex10R | gcactaccaggggtctcaac (SEQ ID NO: 58) | 20 | 59.58 | |
| ADCY6 | ADCY-ex11F | gtgagggcacaggtgtttct (SEQ ID NO: 59) | 20 | 60.16 | 482 |
| ADCY6 | ADCY-ex11R | ctggcatagatcccaagcat (SEQ ID NO: 60) | 20 | 60.06 | |
| ADCY6 | ADCY-ex12F | tgcttcatctgcttcatcca (SEQ ID NO: 61) | 20 | 60.50 | 493 |
| ADCY6 | ADCY-ex12R | agggccttagggaacagcta (SEQ ID NO: 62) | 20 | 60.22 | |
| ADCY6 | ADCY-ex13F | cgtgctgatctgtgctgtgt (SEQ ID NO: 63) | 20 | 61.13 | 498 |
| ADCY6 | ADCY-ex13R | cagggatgacaagaggttgg (SEQ ID NO: 64) | 20 | 60.50 | |
| ADCY6 | ADCY-ex14F | gggaaggttaactccggttc (SEQ ID NO: 65) | 20 | 59.80 | 529 |
| ADCY6 | ADCY-ex14R | cctcaaacccccatatcctc (SEQ ID NO: 66) | 20 | 60.52 | |
| ADCY6 | ADCY-ex15F | ctcccacactaggagcaacc (SEQ ID NO: 67) | 20 | 59.72 | 558 |
| ADCY6 | ADCY-ex15R | tcccaggactagggtttcct (SEQ ID NO: 68) | 20 | 59.93 | |
| ADCY6 | ADCY-ex16F | ggagtactaagagcagaggtat (SEQ ID NO: 69) | 24 | 58.80 | 398 |
| ADCY6 | ADCY-ex16R | cctgctgagcatgcagatac (SEQ ID NO: 70) | 20 | 59.58 | |
| ADCY6 | ADCY-ex17F | cagcaatccaggaagcact (SEQ ID NO: 71) | 19 | 58.97 | 478 |
| ADCY6 | ADCY-ex17R | gtaaagggggcaggtagagg (SEQ ID NO: 72) | 20 | 59.96 | |
| ADCY6 | ADCY-ex18aF | tctgtgcccatcccttactc (SEQ ID NO: 73) | 20 | 60.07 | 395 |
| ADCY6 | ADCY-ex18aR | gcctccagctccacatagaa (SEQ ID NO: 74) | 20 | 60.36 | |
| ADCY6 | ADCY-ex18bF | acaggggagaaggaggagat (SEQ ID NO: 75) | 20 | 59.10 | 439 |
| ADCY6 | ADCY-ex18bR | gctgcctctgacaccttcat (SEQ ID NO: 76) | 20 | 60.42 | |
| ADCY6 | ADCY-ex19F | ggagtcctaggctgcctgtt (SEQ ID NO: 77) | 20 | 60.79 | 559 |
| ADCY6 | ADCY-ex19R | ggacagtgcctaatgatctctg (SEQ ID NO: 78) | 22 | 58.82 | |
| ADCY6 | ADCY-ex20F | ttttgtaaagatagggtttcacca (SEQ ID NO: 79) | 24 | 59.00 | 498 |
| ADCY6 | ADCY-ex20R | aatccttcctgctgcatttg (SEQ ID NO: 80) | 20 | 60.21 | |
| ADCY6 | ADCY-ex21F | ccacaggtgtctctgcagtc (SEQ ID NO: 81) | 20 | 59.44 | 476 |
| ADCY6 | ADCY-ex21R | ccttgttttccagcttgagg (SEQ ID NO: 82) | 20 | 59.85 | |
| CNTNAP1 | CNTNAP-ex1F | gaagggtgggtaaggaggaga (SEQ ID NO: 83) | 21 | 61.71 | 371 |
| CNTNAP1 | CNTNAP-ex1R | cagcctcagagctgcaatg (SEQ ID NO: 84) | 19 | 60.87 | |
| CNTNAP1 | CNTNAP-ex2F | ggagaaggtagcctcctctga (SEQ ID NO: 85) | 21 | 59.96 | 491 |
| CNTNAP1 | CNTNAP-ex2R | ggatccggtgcttcttcat (SEQ ID NO: 86) | 19 | 60.03 | |
| CNTNAP1 | CNTNAP-ex3F | gggcgcctcctcctactac (SEQ ID NO: 87) | 19 | 61.13 | 554 |
| CNTNAP1 | CNTNAP-ex3R | ccctgtgaaccgagtgtttc (SEQ ID NO: 88) | 20 | 60.55 | |
| CNTNAP1 | CNTNAP-ex4F | gggaggctgaggaagagaat (SEQ ID NO: 89) | 20 | 59.78 | 500 |
| CNTNAP1 | CNTNAP-ex4R | tcttgaagctgaaggcgaac (SEQ ID NO: 90) | 20 | 60.66 | |
| CNTNAP1 | CNTNAP-ex5F | ctgcacttccacttcactgc (SEQ ID NO: 91) | 20 | 59.62 | 562 |
| CNTNAP1 | CNTNAP-ex5R | tcactcaactccagtgttttgc (SEQ ID NO: 92) | 22 | 60.33 | |
| CNTNAP1 | CNTNAP-ex6F | gccaggctaaatggcttacc (SEQ ID NO: 93) | 20 | 60.95 | 575 |
| CNTNAP1 | CNTNAP-ex6R | tcactatgttgcccaggtga (SEQ ID NO: 94) | 20 | 60.11 | |
| CNTNAP1 | CNTNAP-ex7F | agccgatttgtgttggagtc (SEQ ID NO: 95) | 20 | 60.12 | 545 |
| CNTNAP1 | CNTNAP-ex7R | cccgaccactgcttcttaac (SEQ ID NO: 96) | 20 | 59.73 | |
| CNTNAP1 | CNTNAP-ex8aF | acagatggacgagcttggag (SEQ ID NO: 97) | 20 | 60.41 | 390 |
| CNTNAP1 | CNTNAP-ex8aR | acacgttgacctgcccttc (SEQ ID NO: 98) | 19 | 61.13 | |
| CNTNAP1 | CNTNAP-ex8bF | agggtaaggtggcttttcgt (SEQ ID NO: 99) | 20 | 60.00 | 450 |
| CNTNAP1 | CNTNAP-ex8bR | ctcagattcgcctcctcact (SEQ ID NO: 100) | 20 | 59.55 | |
| CNTNAP1 | CNTNAP-ex9F | gggtcatggataatgaagagaca (SEQ ID NO: 101) | 23 | 60.20 | 500 |
| CNTNAP1 | CNTNAP-ex9R | agaaaggggactacaaccaaagt(SEQ ID NO: 102) | 23 | 59.47 | |
| CNTNAP1 | CNTNAP-ex10F | gccacatggagaattttgga (SEQ ID NO: 103) | 20 | 60.84 | 560 |
| CNTNAP1 | CNTNAP-ex10R | tgtcatttccatcctgagca (SEQ ID NO: 104) | 20 | 60.20 | |
| CNTNAP1 | CNTNAP-ex11F | agccctgaggactgcctagt (SEQ ID NO: 105) | 20 | 60.41 | 462 |
| CNTNAP1 | CNTNAP-ex11R | gatggcaccactgcactcta (SEQ ID NO: 106) | 20 | 59.86 | |
| CNTNAP1 | CNTNAP-ex12F | ctacaggtgcacaccaccac (SEQ ID NO: 107) | 20 | 60.07 | 540 |
| CNTNAP1 | CNTNAP-ex12R | cgagtagcttcccaaagtgc (SEQ ID NO: 108) | 20 | 60.02 | |
| CNTNAP1 | CNTNAP-ex13F | cccattccagctatcttatttcc (SEQ ID NO: 109) | 23 | 60.17 | 549 |
| CNTNAP1 | CNTNAP-ex13R | aggccctgtccctccatt (SEQ ID NO: 110) | 18 | 61.83 | |
| CNTNAP1 | CNTNAP-ex14F | cggaactggaggagacacc (SEQ ID NO: 111) | 19 | 60.66 | 500 |
| CNTNAP1 | CNTNAP-ex14R | gaagtggagcggttcgtatc (SEQ ID NO: 112) | 20 | 59.70 | |
| CNTNAP1 | CNTNAP-ex15F | cgaaatgaggagcagcactt (SEQ ID NO: 113) | 20 | 60.54 | 528 |
| CNTNAP1 | CNTNAP-ex15R | cggaggggagactaaggagt (SEQ ID NO: 114) | 20 | 59.69 | |
| CNTNAP1 | CNTNAP-ex16F | tgcgatctcattacccctct (SEQ ID NO: 115) | 20 | 59.65 | 550 |
| CNTNAP1 | CNTNAP-ex16R | caagtgaatgcctagggtca (SEQ ID NO: 116) | 20 | 58.72 | |
| CNTNAP1 | CNTNAP-ex17F | ggggagagacaaagggtatga (SEQ ID NO: 117) | 21 | 60.31 | 589 |
| CNTNAP1 | CNTNAP-ex17R | ccaagagggctggaagaata (SEQ ID NO: 118) | 20 | 59.26 | |
| CNTNAP1 | CNTNAP-ex18aF | ccgtgcccagctaattttt (SEQ ID NO: 119) | 19 | 60.08 | 400 |
| CNTNAP1 | CNTNAP-ex18aR | ccgtgaggtcacagtcacac (SEQ ID NO: 120) | 20 | 60.21 | |
| CNTNAP1 | CNTNAP-ex18bF | ctcccaaagtgctgggatta (SEQ ID NO: 121) | 20 | 60.07 | 499 |
| CNTNAP1 | CNTNAP-ex18bR | cctttggagtcacccatgat (SEQ ID NO: 122) | 20 | 59.78 | |
| CNTNAP1 | CNTNAP-ex19aF | ttgtaaaatgcaggggttgg (SEQ ID NO: 123) | 20 | 60.72 | 498 |
| CNTNAP1 | CNTNAP-ex19aR | ggtgctgaagctgaaggaga (SEQ ID NO: 124) | 20 | 60.68 | |
| CNTNAP1 | CNTNAP-ex19bF | agggagttctcccacatgct (SEQ ID NO: 125) | 20 | 61.05 | 498 |
| CNTNAP1 | CNTNAP-ex19bR | tggtcgagtggttagctggt (SEQ ID NO: 126) | 20 | 60.71 | |
| CNTNAP1 | CNTNAP-ex20F | cgtgactacatggctgtgct (SEQ ID NO: 127) | 20 | 59.93 | 483 |
| CNTNAP1 | CNTNAP-ex20R | gcctattgtccccttggag (SEQ ID NO: 128) | 19 | 59.49 | |
| CNTNAP1 | CNTNAP-ex21F | tgcccacttcagattgagc (SEQ ID NO: 129) | 19 | 59.93 | 483 |
| CNTNAP1 | CNTNAP-ex21R | cgggtcaatgactcctgtct (SEQ ID NO: 130) | 20 | 60.11 | |
| CNTNAP1 | CNTNAP-ex22aF | cgcgttttaggcaaggag (SEQ ID NO: 131) | 18 | 59.56 | 400 |
| CNTNAP1 | CNTNAP-ex22aR | cagggatcaagctcaggtg (SEQ ID NO: 132) | 19 | 59.35 | |
| CNTNAP1 | CNTNAP-ex22bF | acaacaccccaggtttctca (SEQ ID NO: 133) | 20 | 60.40 | 400 |
| CNTNAP1 | CNTNAP-ex22bR | tcttgggaggtcagcttagtg (SEQ ID NO: 134) | 21 | 59.48 | |
| CNTNAP1 | CNTNAP-ex23F | cgaatctaattgcggagcta (SEQ ID NO: 135) | 20 | 58.16 | 398 |
| CNTNAP1 | CNTNAP-ex23R | ctctgcccagttatatggca (SEQ ID NO: 136) | 20 | 58.75 | |
| CNTNAP1 | CNTNAP-ex24F | ggtgagatcccaaagatctgaa (SEQ ID NO: 137) | 22 | 60.44 | 508 |
| CNTNAP1 | CNTNAP-ex24R | ttgggtcccatctgtagctc (SEQ ID NO: 138) | 20 | 60.07 | |

1. A method of identifying a subject having or at risk of having or developing an arthrogryposis multiplex congenita (AMC) and/or congenital peripheral neuropathy, comprising determining, in a sample obtained from said subject, the presence or absence of a single nucleotide variant (SNV) located in CNTNAP1, LGI4, ADCY6 or LMOD3 gene.

2. The method according to claim 1, wherein the SNV is selected from the group consisting of CNTNAP1: NM_{—}003632:c.2901_{—}2902del, CNTNAP1: NM_{—}003632:c.3009_{—}3010insT; CNTNAP1: NM_{—}003632:c.2993-2_{—}2994del, LGI4: c.G793A, ADCY6: NM_{—}015270:c.C3346T, LMOD3: NM_{—}198271:c.135_{—}136 insC and wherein:
the presence of the allele (del) of CNTNAP1: NM_{—}003632:c.2901_{—}2902del indicates a high risk of having or developing an arthrogryposis multiplex congenita and/or congenital peripheral neuropathy;
the presence of the allele (insT) of CNTNAP1: NM_{—}003632:c.3009_{—}3010insT indicates a high risk of having or developing an arthrogryposis multiplex congenita and/or congenital peripheral neuropathy;
the presence of the allele (del) of CNTNAP1: NM_{—}003632:c.2993-2_{—}2994del indicates a high risk of having or developing an arthrogryposis multiplex congenita and/or congenital peripheral neuropathy;
the presence of the allele (A) of LGI4: NM_{—}139284:c.G793A indicates a high risk of having or developing an arthrogryposis multiplex congenita and/or congenital peripheral neuropathy;
the presence of the allele (T) of ADCY6: NM_{—}015270:c.C3346T indicates a high risk of having or developing an arthrogryposis multiplex congenita and/or congenital peripheral neuropathy;
the presence of the allele (insC) of LMOD3: NM_{—}198271:c.135_{—}136 insC indicates a high risk of having or developing an arthrogryposis multiplex congenita and/or congenital peripheral neuropathy.

3. The method according to claim 1, wherein the sample is a blood, amniotic fluid or chorionic villi sample.

4. The method according to claim 1, wherein the presence or absence of said SNV is determined by nucleic acid sequencing or by PCR analysis.

5. The method according to claim 1, which comprises a further step of determining the presence or absence of a single nucleotide variant (SNV) in genes known to be responsible for AMC described in table 1 in a sample obtained from said subject

6. A kit for identifying whether a subject has or is at risk of having or developing an arthrogryposis multiplex congenita and/or congenital peripheral neuropathy), comprising:
at least a means for detecting the SNV located in CNTNAP1, LGI4 ADCY6 and LMOD3 and
instructions for use.

7. A kit according to claim 6, comprising:
at least a means for detecting the SNV selected from the group consisting of CNTNAP1: NM_{—}003632:c.2901_{—}2902del, CNTNAP1: NM_{—}003632:c.3009_{—}3010 insT; CNTNAP1: NM_{—}003632:c.2993-2_{—}2994del, LGI4: NM_{—}139284:c.G793A, ADCY6: NM_{—}015270:c.C3346T, LMOD3: NM_{—}198271:c.135_{—}136insC and
instructions for use.

8. A kit according to claim 7, comprising:
at least one primer and/or at least one probe for amplification of a sequence comprising a SNV consisting of CNTNAP1: NM_{—}003632:c.2901_{—}2902del, CNTNAP1: NM_{—} 003632:c.3009_{—}3010insT; CNTNAP1: NM_{—}003632:c.2993-2_{—}2994del, LGI4: NM_{—}139284:c.G793A, ADCY6: NM_{—}015270:c.C3346T, LMOD3: NM_{—}198271:c.135_{—}136insC,
instructions for use.

9. A method for treating arthrogryposis multiplex congenita and/or congenital peripheral neuropathy and/or preventing progression of arthrogryposis multiplex congenita and/or congenital peripheral neuropathy in a patient, wherein the presence of SNV in CNTNAP1, LGI4, ADCY6 and LMOD3 genes in a sample previously obtained from said patient, have been detected by a method according to claim 1, comprising administering a therapeutically effective amount of a nuclease.

10. The method according to claim 9, wherein the SNV is selected from the group consisting of CNTNAP1: NM_{—}003632:c.2901_{—}2902del, CNTNAP1: NM_{—}003632:c.3009_{—}3010 insT; CNTNAP1: NM_{—}003632:c.2993-2_{—}2994del, LGI4: NM_{—}139284:c.G793A, ADCY6: NM_{—}015270:c.C3346T, LMOD3: NM_{—}198271:c.135_{—}136insC and wherein:
the presence of the allele (del) of CNTNAP1: NM_{—}003632:c.2901_{—}2902del is indicative of an arthrogryposis multiplex congenita and/or congenital peripheral neuropathy,
the presence of the allele (insT) of CNTNAP1: NM_{—}003632:c.3009_{—}3010 insT is indicative of an arthrogryposis multiplex congenita and/or congenital peripheral neuropathy;
the presence of the allele (del) of CNTNAP1: NM_{—}003632:c.2993-2_{—}2994del is indicative of an arthrogryposis multiplex congenita and/or congenital peripheral neuropathy;
the presence of the allele (A) of LGI4: NM_{—}139284:c.G793A is indicative of an arthrogryposis multiplex congenita and/or congenital peripheral neuropathy;
the presence of the allele (T) of ADCY6: NM_{—}015270:c.C3346T is indicative of an arthrogryposis multiplex congenita and/or congenital peripheral neuropathy;
the presence of the allele (insC) of LMOD3: NM_{—}198271:c.135_{—}136 insC is indicative of an arthrogryposis multiplex congenita and/or congenital peripheral neuropathy.


SEQUENCE LISTING (138 sequences)

SEQ ID NO: 1 - DNA, 22 bp, Artificial Sequence
  Synthetic primer
aagacatacgaacagggaggaa

SEQ ID NO: 2 - DNA, 20 bp, Artificial Sequence
  Synthetic primer
cttctgctccttcccagatg

SEQ ID NO: 3 - DNA, 20 bp, Artificial Sequence
  Synthetic primer
gaggagggttgagtgctgtc

SEQ ID NO: 4 - DNA, 22 bp, Artificial Sequence
  Synthetic primer
agcccccatctgacattttaac

SEQ ID NO: 5 - DNA, 22 bp, Artificial Sequence
  Synthetic primer
gggagtgtatgctcacacagat

SEQ ID NO: 6 - DNA, 22 bp, Artificial Sequence
  Synthetic primer
ttcatcagtatcacccaaagca

SEQ ID NO: 7 - DNA, 22 bp, Artificial Sequence
  Synthetic primer
cacagtgaaactggcaatgact

SEQ ID NO: 8 - DNA, 22 bp, Artificial Sequence
  Synthetic primer
tcacacatgagtaagggtgtcc

SEQ ID NO: 9 - DNA, 22 bp, Artificial Sequence
  Synthetic primer
gcttcatcgaggacaatgagat

SEQ ID NO: 10 - DNA, 18 bp, Artificial Sequence
  Synthetic primer
cccacctcaggcaagtgt

SEQ ID NO: 11 - DNA, 18 bp, Artificial Sequence
  Synthetic primer
gtgcccacttctggttgg

SEQ ID NO: 12 - DNA, 22 bp, Artificial Sequence
  Synthetic primer
cccatggcactcctatatcact

SEQ ID NO: 13 - DNA, 25 bp, Artificial Sequence
  Synthetic primer
caaacatctacagatcaagattcca

SEQ ID NO: 14 - DNA, 18 bp, Artificial Sequence
  Synthetic primer
cccttccagccacaggag

SEQ ID NO: 15 - DNA, 20 bp, Artificial Sequence
  Synthetic primer
gtgtcctgcaagccactggt

SEQ ID NO: 16 - DNA, 20 bp, Artificial Sequence
  Synthetic primer
atgtagcgtgtgaggcacag

SEQ ID NO: 17 - DNA, 20 bp, Artificial Sequence
  Synthetic primer
ggcttttacccgcaccagag

SEQ ID NO: 18 - DNA, 22 bp, Artificial Sequence
  Synthetic primer
atttgggggtagaatcaggatg

SEQ ID NO: 19 - DNA, 22 bp, Artificial Sequence
  Synthetic primer
aaggtgaaactgacagagtcca

SEQ ID NO: 20 - DNA, 22 bp, Artificial Sequence
  Synthetic primer
ccttaaagcaagcagcaaagag

SEQ ID NO: 21 - DNA, 20 bp, Artificial Sequence
  Synthetic primer
gtcttccagccactgctcat

SEQ ID NO: 22 - DNA, 22 bp, Artificial Sequence
  Synthetic primer
agtaggctgggaccctctatgt

SEQ ID NO: 23 - DNA, 26 bp, Artificial Sequence
  Synthetic primer
aaatatctcaagggtgtgttaacttg

SEQ ID NO: 24 - DNA, 21 bp, Artificial Sequence
  Synthetic primer
cccaattttccccttgtaaat

SEQ ID NO: 25 - DNA, 24 bp, Artificial Sequence
  Synthetic primer
aactgcttctgaaattaaacacca

SEQ ID NO: 26 - DNA, 20 bp, Artificial Sequence
  Synthetic primer
cactttgttcttgggcctct

SEQ ID NO: 27 - DNA, 20 bp, Artificial Sequence
  Synthetic primer
tgatgatgacgacgaaggag

SEQ ID NO: 28 - DNA, 20 bp, Artificial Sequence
  Synthetic primer
ccaacatgtgcctctgattg

SEQ ID NO: 29 - DNA, 20 bp, Artificial Sequence
  Synthetic primer
caatgtgggtgcagatgaga

SEQ ID NO: 30 - DNA, 20 bp, Artificial Sequence
  Synthetic primer
gcgatggctttttcatcatt

SEQ ID NO: 31 - DNA, 19 bp, Artificial Sequence
  Synthetic primer
gaggacccaagccagattc

SEQ ID NO: 32 - DNA, 20 bp, Artificial Sequence
  Synthetic primer
gagatggggtgctacaggtg

SEQ ID NO: 33 - DNA, 20 bp, Artificial Sequence
  Synthetic primer
ggggatacccttgttgccta

SEQ ID NO: 34 - DNA, 20 bp, Artificial Sequence
  Synthetic primer
gtcacctcggtatcctcgaa

SEQ ID NO: 35 - DNA, 20 bp, Artificial Sequence
  Synthetic primer
gtggatgaacggaaaacagc

SEQ ID NO: 36 - DNA, 18 bp, Artificial Sequence
  Synthetic primer
gtggaaagccagcagcac

SEQ ID NO: 37 - DNA, 20 bp, Artificial Sequence
  Synthetic primer
ttcgaggataccgaggtgac

SEQ ID NO: 38 - DNA, 20 bp, Artificial Sequence
  Synthetic primer
aggagcgtgtaggcgatgta

SEQ ID NO: 39 - DNA, 18 bp, Artificial Sequence
  Synthetic primer
gtggcactgttggcctgt

SEQ ID NO: 40 - DNA, 19 bp, Artificial Sequence
  Synthetic primer
agacccctcaaccctagcc

SEQ ID NO: 41 - DNA, 20 bp, Artificial Sequence
  Synthetic primer
cctgtgggtaatgggagaga

SEQ ID NO: 42 - DNA, 19 bp, Artificial Sequence
  Synthetic primer
tccttcccttggacaggac

SEQ ID NO: 43 - DNA, 19 bp, Artificial Sequence
  Synthetic primer
tcaatggagggtggtcaga

SEQ ID NO: 44 - DNA, 21 bp, Artificial Sequence
  Synthetic primer
caaagagctcattcagggtca

SEQ ID NO: 45 - DNA, 20 bp, Artificial Sequence
  Synthetic primer
ttgccatggagatgaaagaa

SEQ ID NO: 46 - DNA, 20 bp, Artificial Sequence
  Synthetic primer
tgaatgggaaggaattggag

SEQ ID NO: 47 - DNA, 18 bp, Artificial Sequence
  Synthetic primer
gagctctttgcccggttt

SEQ ID NO: 48 - DNA, 20 bp, Artificial Sequence
  Synthetic primer
cagatggcattccctctagc

SEQ ID NO: 49 - DNA, 20 bp, Artificial Sequence
  Synthetic primer
tgctgttgaacacagcctta

SEQ ID NO: 50 - DNA, 20 bp, Artificial Sequence
  Synthetic primer
aatgtgctgctccttgaggt

SEQ ID NO: 51 - DNA, 20 bp, Artificial Sequence
  Synthetic primer
aggctggtacgtgaggtgac

SEQ ID NO: 52 - DNA, 20 bp, Artificial Sequence
  Synthetic primer
atcagcccttccatggagtt

SEQ ID NO: 53 - DNA, 20 bp, Artificial Sequence
  Synthetic primer
gtacctcaaggagcagcaca

SEQ ID NO: 54 - DNA, 20 bp, Artificial Sequence
  Synthetic primer
caacactgtgctgagggtgt

SEQ ID NO: 55 - DNA, 20 bp, Artificial Sequence
  Synthetic primer
tgtgactgccatttccatgt

SEQ ID NO: 56 - DNA, 20 bp, Artificial Sequence
  Synthetic primer
ggccatgtgagaaacaatga

SEQ ID NO: 57 - DNA, 20 bp, Artificial Sequence
  Synthetic primer
gcattgatgattccagcaaa

SEQ ID NO: 58 - DNA, 20 bp, Artificial Sequence
  Synthetic primer
gcactaccaggggtctcaac

SEQ ID NO: 59 - DNA, 20 bp, Artificial Sequence
  Synthetic primer
gtgagggcacaggtgtttct

SEQ ID NO: 60 - DNA, 20 bp, Artificial Sequence
  Synthetic primer
ctggcatagatcccaagcat

SEQ ID NO: 61 - DNA, 20 bp, Artificial Sequence
  Synthetic primer
tgcttcatctgcttcatcca

SEQ ID NO: 62 - DNA, 20 bp, Artificial Sequence
  Synthetic primer
agggccttagggaacagcta

SEQ ID NO: 63 - DNA, 20 bp, Artificial Sequence
  Synthetic primer
cgtgctgatctgtgctgtgt

SEQ ID NO: 64 - DNA, 20 bp, Artificial Sequence
  Synthetic primer
cagggatgacaagaggttgg

SEQ ID NO: 65 - DNA, 20 bp, Artificial Sequence
  Synthetic primer
gggaaggttaactccggttc

SEQ ID NO: 66 - DNA, 20 bp, Artificial Sequence
  Synthetic primer
cctcaaacccccatatcctc

SEQ ID NO: 67 - DNA, 20 bp, Artificial Sequence
  Synthetic primer
ctcccacactaggagcaacc

SEQ ID NO: 68 - DNA, 20 bp, Artificial Sequence
  Synthetic primer
tcccaggactagggtttcct

SEQ ID NO: 69 - DNA, 24 bp, Artificial Sequence
  Synthetic primer
ggagtctctaagagcagaggtttt

SEQ ID NO: 70 - DNA, 20 bp, Artificial Sequence
  Synthetic primer
cctgctgagcatgcagatac

SEQ ID NO: 71 - DNA, 19 bp, Artificial Sequence
  Synthetic primer
cagcaatccaggaagcact

SEQ ID NO: 72 - DNA, 20 bp, Artificial Sequence
  Synthetic primer
gtaaagggggcaggtagagg

SEQ ID NO: 73 - DNA, 20 bp, Artificial Sequence
  Synthetic primer
tctgtgcccatcccttactc

SEQ ID NO: 74 - DNA, 20 bp, Artificial Sequence
  Synthetic primer
gcctccagctccacatagaa

SEQ ID NO: 75 - DNA, 20 bp, Artificial Sequence
  Synthetic primer
acaggggagaaggaggagat

SEQ ID NO: 76 - DNA, 20 bp, Artificial Sequence
  Synthetic primer
gctgcctctgacaccttcat

SEQ ID NO: 77 - DNA, 20 bp, Artificial Sequence
  Synthetic primer
ggagtcctaggctgcctgtt

SEQ ID NO: 78 - DNA, 22 bp, Artificial Sequence
  Synthetic primer
ggacagtgcctaatgatctctg

SEQ ID NO: 79 - DNA, 24 bp, Artificial Sequence
  Synthetic primer
ttttgtaaagatagggtttcacca

SEQ ID NO: 80 - DNA, 20 bp, Artificial Sequence
  Synthetic primer
aatccttcctgctgcatttg

SEQ ID NO: 81 - DNA, 20 bp, Artificial Sequence
  Synthetic primer
ccacaggtgtctctgcagtc

SEQ ID NO: 82 - DNA, 20 bp, Artificial Sequence
  Synthetic primer
ccttgttttccagcttgagg

SEQ ID NO: 83 - DNA, 21 bp, Artificial Sequence
  Synthetic primer
gaagggtgggtaaggaggaga

SEQ ID NO: 84 - DNA, 19 bp, Artificial Sequence
  Synthetic primer
cagcctcagagctgcaatg

SEQ ID NO: 85 - DNA, 21 bp, Artificial Sequence
  Synthetic primer
ggagaaggtagcctcctctga

SEQ ID NO: 86 - DNA, 19 bp, Artificial Sequence
  Synthetic primer
ggatccggtgcttcttcat

SEQ ID NO: 87 - DNA, 19 bp, Artificial Sequence
  Synthetic primer
gggcgcctcctcctactac

SEQ ID NO: 88 - DNA, 20 bp, Artificial Sequence
  Synthetic primer
ccctgtgaaccgagtgtttc

SEQ ID NO: 89 - DNA, 20 bp, Artificial Sequence
  Synthetic primer
gggaggctgaggaagagaat

SEQ ID NO: 90 - DNA, 20 bp, Artificial Sequence
  Synthetic primer
tcttgaagctgaaggcgaac

SEQ ID NO: 91 - DNA, 20 bp, Artificial Sequence
  Synthetic primer
ctgcacttccacttcactgc

SEQ ID NO: 92 - DNA, 22 bp, Artificial Sequence
  Synthetic primer
tcactcaactccagtgttttgc

SEQ ID NO: 93 - DNA, 20 bp, Artificial Sequence
  Synthetic primer
gccaggctaaatggcttacc

SEQ ID NO: 94 - DNA, 20 bp, Artificial Sequence
  Synthetic primer
tcactatgttgcccaggtga

SEQ ID NO: 95 - DNA, 20 bp, Artificial Sequence
  Synthetic primer
agccgatttgtgttggagtc

SEQ ID NO: 96 - DNA, 20 bp, Artificial Sequence
  Synthetic primer
cccgaccactgcttcttaac

SEQ ID NO: 97 - DNA, 20 bp, Artificial Sequence
  Synthetic primer
acagatggacgagcttggag

SEQ ID NO: 98 - DNA, 19 bp, Artificial Sequence
  Synthetic primer
acacgttgacctgcccttc

SEQ ID NO: 99 - DNA, 20 bp, Artificial Sequence
  Synthetic primer
agggtaaggtggcttttcgt

SEQ ID NO: 100 - DNA, 20 bp, Artificial Sequence
  Synthetic primer
ctcagattcgcctcctcact

SEQ ID NO: 101 - DNA, 23 bp, Artificial Sequence
  Synthetic primer
gggtcatggataatgaagagaca

SEQ ID NO: 102 - DNA, 23 bp, Artificial Sequence
  Synthetic primer
agaaaggggactacaaccaaagt

SEQ ID NO: 103 - DNA, 20 bp, Artificial Sequence
  Synthetic primer
gccacatggagaattttgga

SEQ ID NO: 104 - DNA, 20 bp, Artificial Sequence
  Synthetic primer
tgtcatttccatcctgagca

SEQ ID NO: 105 - DNA, 20 bp, Artificial Sequence
  Synthetic primer
agccctgaggactgcctagt

SEQ ID NO: 106 - DNA, 20 bp, Artificial Sequence
  Synthetic primer
gatggcaccactgcactcta

SEQ ID NO: 107 - DNA, 20 bp, Artificial Sequence
  Synthetic primer
ctacaggtgcacaccaccac

SEQ ID NO: 108 - DNA, 20 bp, Artificial Sequence
  Synthetic primer
cgagtagcttcccaaagtgc

SEQ ID NO: 109 - DNA, 23 bp, Artificial Sequence
  Synthetic primer
cccattccagctatcttatttcc

SEQ ID NO: 110 - DNA, 18 bp, Artificial Sequence
  Synthetic primer
aggccctgtccctccatt

SEQ ID NO: 111 - DNA, 19 bp, Artificial Sequence
  Synthetic primer
cggaactggaggagacacc

SEQ ID NO: 112 - DNA, 20 bp, Artificial Sequence
  Synthetic primer
gaagtggagcggttcgtatc

SEQ ID NO: 113 - DNA, 20 bp, Artificial Sequence
  Synthetic primer
cgaaatgaggagcagcactt

SEQ ID NO: 114 - DNA, 20 bp, Artificial Sequence
  Synthetic primer
cggaggggagactaaggagt

SEQ ID NO: 115 - DNA, 20 bp, Artificial Sequence
  Synthetic primer
tgcgatctcattacccctct

SEQ ID NO: 116 - DNA, 20 bp, Artificial Sequence
  Synthetic primer
caagtgaatgcctagggtca

SEQ ID NO: 117 - DNA, 21 bp, Artificial Sequence
  Synthetic primer
ggggagagacaaagggtatga

SEQ ID NO: 118 - DNA, 20 bp, Artificial Sequence
  Synthetic primer
ccaagagggctggaagaata

SEQ ID NO: 119 - DNA, 19 bp, Artificial Sequence
  Synthetic primer
ccgtgcccagctaattttt

SEQ ID NO: 120 - DNA, 20 bp, Artificial Sequence
  Synthetic primer
ccgtgaggtcacagtcacac

SEQ ID NO: 121 - DNA, 20 bp, Artificial Sequence
  Synthetic primer
ctcccaaagtgctgggatta

SEQ ID NO: 122 - DNA, 20 bp, Artificial Sequence
  Synthetic primer
cctttggagtcacccatgat

SEQ ID NO: 123 - DNA, 20 bp, Artificial Sequence
  Synthetic primer
ttgtaaaatgcaggggttgg

SEQ ID NO: 124 - DNA, 20 bp, Artificial Sequence
  Synthetic primer
ggtgctgaagctgaaggaga

SEQ ID NO: 125 - DNA, 20 bp, Artificial Sequence
  Synthetic primer
agggagttctcccacatgct

SEQ ID NO: 126 - DNA, 20 bp, Artificial Sequence
  Synthetic primer
tggtcgagtggttagctggt

SEQ ID NO: 127 - DNA, 20 bp, Artificial Sequence
  Synthetic primer
cgtgactacatggctgtgct

SEQ ID NO: 128 - DNA, 19 bp, Artificial Sequence
  Synthetic primer
gcctattgtccccttggag

SEQ ID NO: 129 - DNA, 19 bp, Artificial Sequence
  Synthetic primer
tgcccacttcagattgagc

SEQ ID NO: 130 - DNA, 20 bp, Artificial Sequence
  Synthetic primer
cgggtcaatgactcctgtct

SEQ ID NO: 131 - DNA, 18 bp, Artificial Sequence
  Synthetic primer
cgcgttttaggcaaggag

SEQ ID NO: 132 - DNA, 19 bp, Artificial Sequence
  Synthetic primer
cagggatcaagctcaggtg

SEQ ID NO: 133 - DNA, 20 bp, Artificial Sequence
  Synthetic primer
acaacaccccaggtttctca

SEQ ID NO: 134 - DNA, 21 bp, Artificial Sequence
  Synthetic primer
tcttgggaggtcagcttagtg

SEQ ID NO: 135 - DNA, 20 bp, Artificial Sequence
  Synthetic primer
cgaatctaattgcggagcta

SEQ ID NO: 136 - DNA, 20 bp, Artificial Sequence
  Synthetic primer
ctctgcccagttatatggca

SEQ ID NO: 137 - DNA, 22 bp, Artificial Sequence
  Synthetic primer
ggtgagatcccaaagatctgaa

SEQ ID NO: 138 - DNA, 20 bp, Artificial Sequence
  Synthetic primer
ttgggtcccatctgtagctc